(19) United States
(12) Patent Application Publication
(10) Pub. No.: US 2010/0093611 A1
(43) Pub. Date: Apr. 15, 2010

(54) COMPOUNDS AND METHODS FOR TREATING OR PREVENTING AUTOIMMUNE DISEASES

(76) Inventors: Stephen K. Horrigan, Poolesvile, MD (US); Qin Zong, Germantown, MD (US); Daniel Soppet, Mclean, VA (US); Juana Castaneda, Rockville, MD (US); Bo Chen, Frederick, MD (US); Ricardo Cibotti, Bethesta, MD (US); Laurent P. Audoly, Gaithersburg, MD (US); Anthony Coyle, Washington, DC (US); Peter Kiener, Potomac, MD (US)

Correspondence Address: Alan J. Grant, Carella Byrne Bain Gilfillan Cecchi Stewart, 5 Becker Farm, Roseland, NJ, 07068, US

(21) Appl. No.: 12/451,539

(22) PCT Filed: May 16, 2008
(86) PCT No.: PCT/US2008/006345

§ 371 Date: Nov. 16, 2009

Related U.S. Application Data
(60) Provisional application No. 60/930,473, filed on May 16, 2007.
(60) Provisional application No. 61/007,099, filed on Dec. 11, 2007.

(51) Int. Cl. A61K 38/12; A61K 31/435; A61K 31/515; A61K 31/551; A61P 37/06

(52) U.S. Cl. 514/10; 514/296; 514/285; 514/270; 514/217

(57) ABSTRACT

Methods of treating, ameliorating, preventing, or reducing the risk of developing an auto-immune disease and/or an inflammatory condition, such as systemic lupus erythematosus, in a patient, such as a human being, using a therapeutically effective amount of an agent(s) that inhibits the activity of one or more of histone deacetylase (HDAC), I_{K}B kinase (IKK-2), nuclear factor kB (NF-kB), ubiquitin/proteasome and Janus kinase (JAK) are disclosed. Compounds useful in such methods are also presented.

The present application claims priority of U.S. Provisional Applications 60/930,473, filed 16 May 2007, and 61/007,099, filed 11 Dec. 2007, the disclosures of which are hereby incorporated by reference in their entirety.

### FIELD OF THE INVENTION

The present invention relates to treatment of autoimmune diseases and conditions in a patient and caused directly or indirectly by the production of interferons (IFNs) and the production of auto-antibodies to target cells, resulting in damage to the patient's immune system with pathological results.

### BACKGROUND OF THE INVENTION

The immune system is able to discriminate between “self” and “non-self” antigens, which is necessary for defense against sources of foreign (i.e., non-self) antigens such as invading microorganisms. “Non-self” antigens are substances different from the patient's own constituents, while “self” antigens are those that are not detectably different or foreign from a patient's own constituents. Unfortunately, in certain disease states a person's immune system identifies its own otherwise normal constituents as “non-self” and mounts an immune response against what is actually “self” material, with deleterious, possibly severe or even fatal, results. Thus, such conditions are referred to an autoimmune disease and result when a person's immune system attacks his own tissues or organs to produce clinical conditions otherwise associated with the destruction of that tissue or organ. Common autoimmune diseases include insulin-dependent (Type I) diabetes, rheumatic fever, rheumatoid arthritis, acquired immunodeficiency syndrome (“AIDS”), psoriasis, Graves' disease, myasthenia gravis, multiple sclerosis, and systemic lupus erythematosus (SLE)¹⁻⁶.

Autoimmune disease conditions may result from a genetic pre-disposition for such diseases or may be the result of foreign agents, including some viruses. Some such diseases, most notably Type I diabetes, are a result of genetic predisposition, caused by the destruction of the insulin producing beta-cells of the pancreas through an immune cell mediated response. A etymology is seen with diseases like multiple sclerosis (resulting from the destruction of the conducting fibers of the nervous system) and rheumatoid arthritis (where the joint lining tissues are destroyed).

Type I IFNs are a family of pleiotropic cytokines that play an important role in modulating nearly all phases of immune and inflammatory responses^{6, 7}. Type I IFNs include α, β, ε, κ, and ω subtypes expressed by 13 functional IFN-α genes, and single IFN-β, IFN-ε, IFN-κ, and IFN-ω genes⁷. Binding of type I IFNs to a common receptor (IFNAR), composed of a unique IFNAR-1 subunit and a functionally active IFNAR-2c subunit, results in the activation of Jak1 and Tyk2 kinases that subsequently activate the signal transducer and activator of transcription (STAT) proteins 1, 2, 3, 4 and 5 and regulate the expression of hundreds of interferon stimulated genes (ISGs)⁸⁻¹⁰. The cooperative function of several signaling cascades is required for the generation of type I IFNs-mediated responses¹⁰, however, the precise relationships between type I IFNs- and the biochemical functions and signaling pathways they induce have not been fully elucidated.

RNA and DNA fragments from invading microorganisms induce the production of type I IFNs by the activation of Toll-like receptors 3, 7, 8 and 9 that are differentially expressed in epithelial cells, monocytes, dendritic cells (DC) and plasmacytoid dendritic cells (pDC)^{11, 12}. In addition to their critical role in the control of viral replication, type I IFNs expressed by activated pDC contribute to linking the innate and adaptive immune responses to invading pathogens through the induction, differentiation, activation and survival of multiple cell subsets such as monocytes, DC, NK, CD8⁺T and B cells¹³.

Systemic Lupus Erythematosus (SLE) is a chronic, usually life-long, and possibly fatal autoimmune disease, affecting mostly women (especially of child-bearing age) and minority groups in the U.S., and is characterized by variable clinical manifestations. Often, problems with the joints, skin, kidney, brain, lung, heart, and GI tract are involved. In the Unites States today as many as 2 million persons may be afflicted with incidence varying, some areas (like Birmingham, Ala.) showing very high incidence. More recently, the prognosis for such patients has improved, although such improvement may be due more to success in treating the symptoms rather than the causes of the disease.^{4, 14-22}

Other autoimmune diseases result from a variety of causes, including the coincidental cross-reactivity with otherwise foreign antigens. For example, in rheumatic fever an antigen of the streptococcal bacterium that causes rheumatic fever is cross-reactive with antigens found in human heart muscle, so that antibodies against the microbe cannot distinguish very well between the target microorganism and the tissues of the heart, leading to destruction of both.

More recently, a new hypothesis has developed wherein autoimmune diseases (for example, lupus) are related to changes in synthesis of cytokines, such as interferons, especially interferon alpha (IFNα). See, for example, Gringeri et al., J. Acquir. Immun. Defic. Syndr. 13:55-67 (1996)).

Interferons are usually not found in the blood of people other than those infected with a virus and so otherwise healthy individuals should not have interferon on their blood stream. However, these molecules (such as IFNα) have been detected (with antibodies) in the plasma of persons afflicted with autoimmune diseases. For example, patients with HIV infection have detectable amount of IFNα in their circulation (see, for example, DeStefano et al., J. Infec. Disease 146:451 (1982)). In addition, overproduction of interferon is implicated in SLE (see Banchereau et al., Immunity, Vol 25, pp. 383-392 (2006)). Interferons have also been linked to cell signaling and specific target molecules, such as JAK/STAT (see van Boxel-Dezaire et al., Immunity, Vol. 25, pp. 361-372 (2006)). Interaction between interferon and molecular targets such as NF-κB as been reported (see: Pfeffer et al, J. Biol. Chem., 279, 31304-31311 (2004). The mechanism of interferon gene regulation involves acetylation and has been studied (see, for example, Nusinzon and Horvath, J. Interf. Cytok. Res., 25:745-748 (2005)).

Other agents, such as tumor necrosis factors (TNFα and TNFβ), have also been detected in patients with autoimmune disease (especially rheumatoid arthritis (RA)(see, for example, Brennan et al., Brit. J. Rheum. 31(5):293-8 (1992)) and in, in experimental animals, in some cases of arthritis (Brahn et al., Lymphokine and Cytokine Res. 11 (5):253 (1992)). TNF-α is elevated in the blood of rheumatoid arthritis patients (Altomonte et al., Clin. Rheum. 11 (2):202 (1992).

While multiple lines of evidence indicate that inhibition of Type I interferons (IFNs), including IFNα, may afford a therapeutic benefit in treating a number of autoimmune diseases, such as SLE, attempts at taking advantage of this approach have been limited to such mechanisms as use of interferon receptors and anti-interferon antibodies (see, for example, U.S. Pat. No. 6,333,032, utilizing anti-IFNγ antibodies to treat auto-immune diseases such as arthritis). Because antibodies are relatively large molecules that may themselves induce an immune response, there remains a need to utilize other types of agents in treating such diseases, preferably small organic compounds. Current treatment of autoimmune diseases relies mostly on use of immunosuppressive agents such as cortisone, methotrexate, cyclophosphamide and the like but all of which suffer the drawback of inhibiting the immune system and thereby rendering the patient highly susceptible to other diseases, especially infectious diseases.

The deleterious effect of a deregulated expression of IFN-α, is not uniquely restricted to SLE. For example, in a xenograft model of human psoriasis, blocking IFN-α signaling or inhibiting the ability of pDCs to produce IFN-α prevented the T cell-dependent development of psoriasis²³. A number of reports correlate IFN-α2b therapeutic treatment and development of dermatomyositis²⁴. Altogether, these data strongly indicate that targeting the IFN-α pathway may provide an effective approach for the treatment of SLE, and other autoimmune disorders associated with dysregulation of type I IFN signaling pathways such as psoriasis, type I diabetes, Sjögren's disease, and inflammatory myopathies²⁵.

Therapeutic modulation across the spectrum of type I IFNs pathways represents a novel and promising approach which represents a challenge to the conventional single target drug discovery. Recent advances in molecular biology, robotics, and assay detection technologies make it feasible to explore gene, protein, and signaling pathways in an integrated cellular context. This has opened the door to use of molecular profiling, for every step in the drug discovery and development process^{26, 27}. Molecular profiling by these approaches has several potential advantages both as a primary anchor to drug discovery and as a complement to more conventional target-based discovery efforts. The use of large complex sets of genomic biomarkers has already found its way into standard use in the identification and validation of drug targets^{26, 27}. Profiling the expression of large gene sets in normal compared to disease states can provide critical clues to the activities of cellular control pathways as well as identifying specific gene signatures as the surrogate markers in disease processes. An exciting use of such molecular surrogate markers that has the potential to revolutionize drug discovery is its utility in defining cellular states as the primary driver for the identification of drug candidates²⁸⁻²⁰.

The present invention provides a new approach to treating autoimmune diseases and a new mechanism for identifying potentially successful therapeutic agents for such treatment. Thus, by utilizing high throughput screening procedures relying on selected target genes plus immunological assays in primary human cells, the present invention provides inhibitors of the interferon, for example, IFNα, pathway. The result is a new approach whereby agents targeting a select gene signature have been identified, which compounds inhibit IFNα and are useful in treating auto-immune diseases, especially lupus.

### BRIEF SUMMARY OF THE INVENTION

In one aspect, the present invention relates to a method of treating or ameliorating an auto-immune disease in a mammal, preferably a human being, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In one aspect, the present invention relates to a method of treating or ameliorating an inflammatory condition in a mammal, preferably a human being, comprising administering to a mammal in need thereof a therapeutically effective amount of an agent that inhibits the activity of a molecule selected from the group consisting of histone deacetylase (HDCA), I_{K}B kinase (IKK-2), nuclear factor kB (NF-kB), ubiquitin/proteasome and Janus kinase (JAK).

In another aspect, the present invention relates to a method of preventing or reducing the risk of developing an auto-immune disease in a mammal, preferably a human patient, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In another aspect, the present invention relates to a method of preventing or reducing the risk of developing an inflammatory condition in a mammal, preferably a human patient, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In a preferred embodiment of the methods of the invention, the agent inhibits more than one of said molecules. In separate embodiments, the agent inhibits HDAC, and/or inhibits ubiquitin/proteasome, and/or inhibits nuclear factor kB (NF-kB), and/or inhibits Janus kinase (JAK), and/or inhibits I_{K}B kinase (IKK-2).

In preferred embodiments, the autoimmune disease or inflammatory disorder contemplated for therapeutic intervention is one or more of type I diabetes, rheumatoid arthritis, psoriasis, psoriatic arthritis, ankylosing spondylitis, scleroderma, Reiter's Syndrome, systemic lupus erythematosus, dermatomyositis, Sjogren's syndrome, lupus erythematosus, multiple sclerosis, myasthenia gravis, asthma, encephilitis, inflammatory bowel disease, chronic obstructive pulmonary disease (COPD), arthritis, idiopathic inflammatory myopathies (IIM), dermatomyositis (DM, polymyositis (PM), includion body myositis or an allergic disorder, preferably systemic lupus erythematosus (SLE).

Representative agents for use in the methods of the invention include, but are not limited to, compounds 1-11 of Table 1 and compounds 1-46 of Table 2.

### BRIEF DESCRIPTION OF THE DRAWINGS

FIG. 1 shows identified IFNα gene signature by microarray and the establishment of a chemical genomics-based platform to identify specific inhibitors of the IFN-α pathway. The unique gene expression signature of the IFN-α signaling pathway was established in THP-1 cells by comparing the gene profiles of the fully activated IFNα pathway to the basal gene expression. THP-1 cells were stimulated either with 100 IU/ml IFN-α, 100 IU/ml IFN-γ, or 10 ng/ml TNF-α to generate the activated pathway gene signatures. This method employed six reproducibly up-regulated genes (DDX58 (Accession No. NM_{—}014314), G1P2 (Accession No. NM_{—}005101), IFI35 (Accession No. BC001356), MX1 (Accession No. NM_{—}002462), OAS3 (Accession No. NM_{—}006187) and RSAD2 (Accession No. (AI337069)), one down-regulated gene (HNRPA0—Accession No. BE966599), and a normalization control gene (GAPDH) that were validated and run in a qPCR-based HITS assay. This set of genes was used to screen for inhibitors of IFN-α signaling pathway. Shown is a typical expression profile of the gene set following IFN-α treatment.

FIG. 2 shows identification of the SLE disease state. Human healthy donor PBMCs were stimulated with 50% serum isolated from either SLE patients or healthy donors. Following a 4 h treatment incubation, cells were lysed and RNA was prepared for qPCR analysis using Avalonrx-HITS assay. Relative expression of six IFNα up-regulated genes (MX1, OAS3, DDX58, RSAD2, G1P2 and IFI35) are shown in panels A to F respectively. Each dot represents an individual sample (n=38 for SLE samples, n=11 for healthy donor samples). Horizontal lines show median gene expression values normalized to GAPDH. Statistic comparison between two groups was conducted by simple student t-tests.

FIG. 3 shows the effects of small molecule inhibitors on the IFN-α gene signature. THP-1 cells were treated with serial dilution of three selected compounds in the presence of 100 IU/ml IFN-α and profiled on Affymertrix human U133A chips. TI₅₀ (50% transcription inhibition) values of all IFN-α signature genes were determined and the distributions of the TI₅₀ were plotted.

FIG. 4 shows the dose inhibitory effects of some small molecule inhibitors of Table 1 on SLE-associated gene signature as induced by SLE serum. JAK inhibitor I (A), IKK2 inhibitor IV (B) or Apicidin 1a (C) were preincubated with freshly isolated monocytes from healthy donor for 30 minutes at the indicated concentration before the cells were stimulated with 50% SLE patient serum for four hours. RNA from lysed cells was examined by qPCR analysis in HITS assay. Data shown here are from three individual SLE patients. Percentage inhibition represents the mean inhibition of the six most robustly induced IFN-α signature genes compared to the vehicle control. Each bar represents the average+/−SEM of three independent experiments using serum from different SLE patients.

FIG. 5 shows inhibition of primary human monocyte activation. Freshly isolated monocytes from healthy donor were pre-incubated with compounds or vehicle control for 0.5 hour before stimulation with 500 IU/ml IFN-α. Cell surface expression of CD80, CD123 and CD38 was evaluated by FACS 48 h post-stimulation. Each bar represents the average+/−SEM of three independent experiments.

FIG. 6 shows the effects of inhibitors on In vitro and in vivo chemokine release. Freshly isolated monocytes were treated with 500 IU/ml IFN-α with or without indicated compounds for 48 h. Supernatants were collected and MCP-1 and IP-10 levels were analyzed by Searchlight Inc. Percentage inhibition represents inhibition of the upregulation of chemokine induced by IFN-α compared to the vehicle control. Panel A: Effect of compounds on IP-10 secretion. Panel B: Effect of compounds on MCP-1 secretion. Each bar represents the average+/−SEM of three independent experiments. Panel C: IKK2 inhibitor IV blocked IFN-α induced IP-10 protein level in vivo. Female NZBW/F1 mice were treated with either antibody (i.p. injection 30 mg/kg) or IKK2 inhibitor (45 mg/kg BID) as described in materials and methods. Adenovirus-IFN-a (1×10¹⁰ viral particles) was administrated i.v. at time 0. Samples were collected 6 h post-stimulation, IP-10 protein level was measured by ELISA. Data representative of two experiments.

FIG. 7 shows dissociated anti-viral properties of lead candidates. Hep-2 cells were seeded at 2×10⁴ cells/well in 96 well plates. Cells were infected with HVS1 at an Multiplicity Of Infection (MOI) of 5 for 1 h at 37° C. 24 hours post-seeding. Virus was aspirated, cells were washed, and 200 μl of medium with or without IFN-α or compound were added as indicated. After 48 h, cells were washed, lysed and luciferase activity were measured according to the manufacturer's instructions. Three independent experiments were performed with similar results. Each bar represents the mean+/−SEM of triplicates from one experiment.

### DEFINITIONS

The term “autoimmune disease” refers diseases wherein the body's immune system, which ordinarily prevents infection by agents such as microorganisms like bacteria and viruses, mis-functions and produces antibodies specific for otherwise normal bodily cells, tissues and organs. Such autoimmune reactions can be mediated by antibodies, T cells and macrophages.

The term “patient” generally refers to a mammal, especially a human being, afflicted with, or at risk of developing, an auto-immune disease.

The term “cytokine” refers to intercellular mediators secreted by sells such as the lymphocytes and macrophages and that participate in the generation of the immune response in mammals. Cytokines include interferons and TNFs that can induce production of other cytokines.

The term “interferon” or “IFN” means any known subtype of interferon, which could include any of the subtypes of INF; such as IFNα and IFNγ. The terms “interferon alpha”, “IFN alpha” and “IFNα” are used interchangeably and intended to refer to IFN alpha proteins encoded by a functional gene of the interferon alpha gene locus. Examples of IFN alpha subtypes include IFN alpha 1, alpha 2a, alpha 2b, alpha 4, alpha 5, alpha 6, alpha 7, alpha 8, alpha 10, alpha 13, alpha. 14, alpha 16, alpha 17 and alpha 21.

The term “autoantigen” refers to a patient's self-produced protein or other antigenic molecule that is recognized by that patient's immune system as if the antigen were of foreign origin, resulting in an autoimmune response in the patient, usually involving production of autoantibodies.

The term “autoantibody” means an antibody produced by an autoimmune patient to one or more of his own antigenic molecules that are otherwise perceived to be foreign. For example, in SLE autoantibodies are produced against the patient's own DNA.

The term “therapeutically effective amount” of an agent of the invention is an amount that is effective, upon single or multiple dose administration to a patient, to treat, ameliorate, prevent or reduce the risk of developing an autoimmune disease, especially lupus or an inflammatory disease, as the case may be. Compounds useful in the methods of the invention cause at least a 50% change, preferably at least a 50% decrease, in the maximal change in gene expression that is induced by IFN alpha in at least 30%, preferably 50%, of the genes used in a gene profile, such as the gene profile formed by the 6 genes identified herein. Where therapy is based on gene modulation, the dosage of said compound producing said effect in vivo would be considered a therapeutically effective amount.

### DETAILED DESCRIPTION OF THE INVENTION

The present invention provides methods for treating or preventing autoimmune diseases and/or inflammatory conditions in a mammal by inhibiting selected target molecules, including histone deacetylase (HDAC), I_{K}B kinase (IKK-2), nuclear factor kB (NF-kB), ubiquitin/proteasome and Janus kinase (JAK).

In one aspect, the present invention relates to a method of treating or ameliorating an auto-immune disease in a mammal, preferably a human being, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In another aspect, the present invention relates to a method of preventing or reducing the risk of developing an auto-immune disease and/or inflammatory condition in a mammal, preferably a human patient, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

Examples of known auto-immune diseases include acute disseminated encephalomyelitis (ADEM—a form of encephalitis), Addison's disease (autoimmune destruction of the adrenal cortex), Ankylosing spondylitis (chronic progressive inflammatory arthritis affecting spine and eventually resulting in fusion of the spine), antiphospholipid antibody syndrome (APS, causing blood clots to form in veins and/or arteries), aplastic anemia (autoimmune attack on the bone marrow), autoimmune hepatitis (the immune system targets the liver), autoimmune oophoritis (immune system attacks the female reproductive organs), coeliac disease (a chronic inflammation of the proximal portion of the small intestine), Crohn's disease (inflammatory bowel disease characterized by chronic inflammation of the intestinal tract), Type I diabetes (autoimmune attack on the insulin-producing beta cells in the islets of Langerhans in the pancreas), Goodpasture's syndrome (destruction of the kidneys and the lungs through autoimmune reaction), Graves' disease (hyperthyroidism caused by anti-thyroid antibodies that stimulate overproduction of thyroid hormone), Guillain-Barré Syndrome (GBS—acquired immune-mediated inflammatory disorder of the peripheral nervous system), Hashimoto's Disease (hypothyroidism), Idiopathic thrombocytopenic purpura (body produces anti-platelet antibodies resulting in a low platelet count), Kawasaki's Disease (autoimmune attack on the arteries around the heart), Lupus erythematosus (the immune system becomes hyperactive and attacks normal tissue), Multiple sclerosis (decreased nerve function due to myelin loss), myasthenia gravis (disorder of neuromuscular transmission), Opsoclonus myoclonus syndrome (OMS—autoimmune attack on the nervous system), Optic neuritis (inflammation of the optic nerve), Ord's thyroiditis (similar to Hashimoto's disease), Pemphigus (blistering and sores on skin and mucous membranes), pernicious anemia (anemia due to improper absorption of vitamin B12), polyarthritis, rheumatoid arthritis (immune system attacks the bone joints), scleroderma, Reiter's syndrome (response to a bacterial infection) Sjögren's syndrome (immune cells attack and destroy the exocrine glands that produce tears and saliva), Takayasu's arteritis (narrowing of the lumen of arteries), and Wegener's granulomatosis (inflammation of the lungs and kidneys).

The compounds for use in the methods of the invention may also inhibit interferon-mediated activation of selected genes. These interferons include interferon gamma and interferon alpha, such as interferon 2α. Such methods include identification of selected chemical agents, mostly small organic compounds, that function to inhibit selected target molecules while preferably having no effect on the anti-viral activity of interferons.

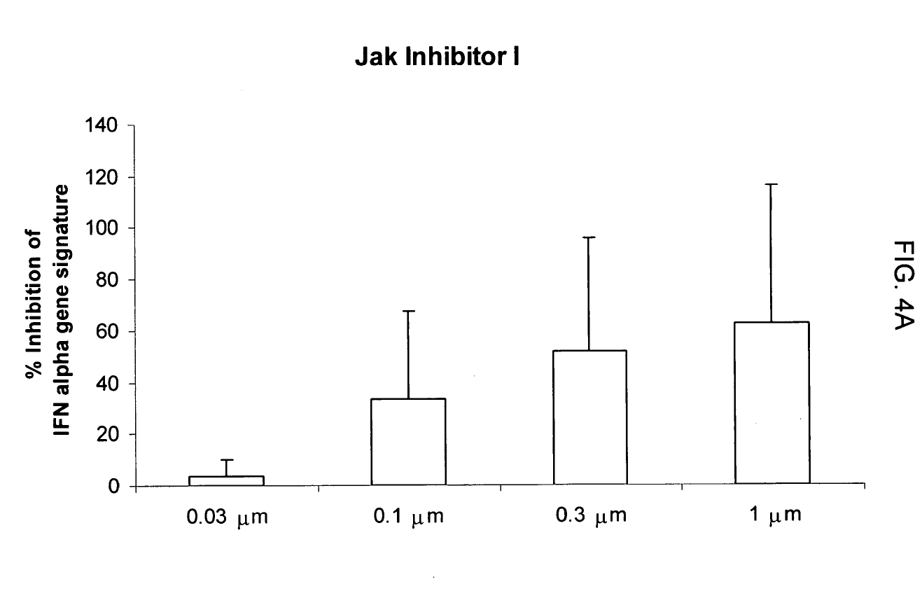

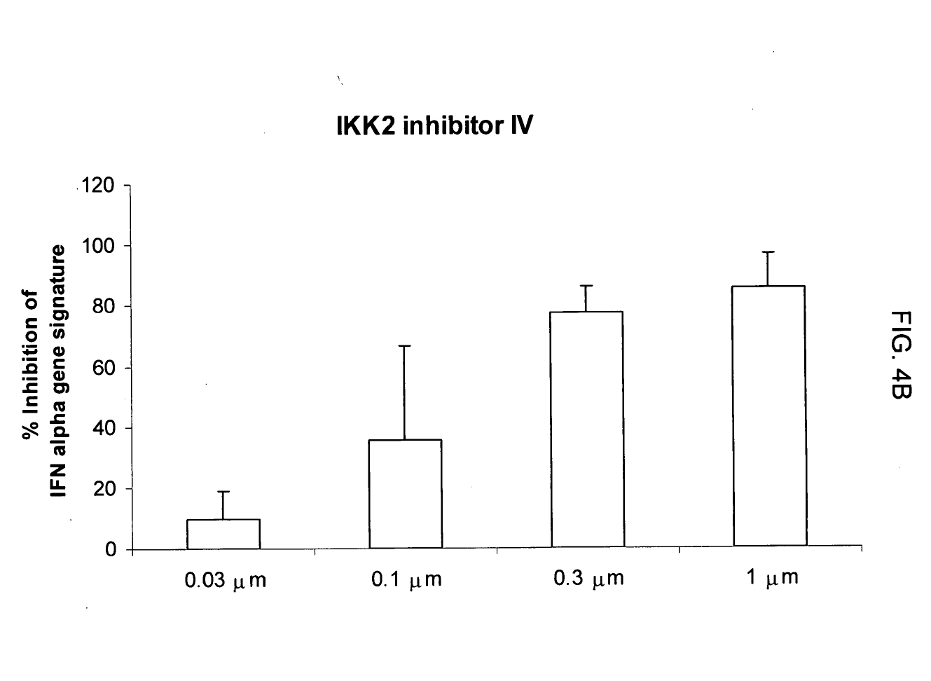

Thus, the compounds disclosed herein blocked both IFNα-induced human monocyte activation and differentiation into dendritic cells and the IFNα-associated gene signature induced by SLE serum in human monocytes. In addition to their dose-dependent anti-inflammatory effects (dee, for example, FIG. 4), inhibitors targeting NF-kB or JAK/STAT signaling did not modulate IFNα anti-viral effects in an in vitro HSV-1 replication assay.

The compounds disclosed herein for use in the methods of the invention inhibit the purified target molecule proteins, such as JAK/STAT and NF-kB in an in vitro assay. Such in vitro assays are known in the art.

In another aspect, the present invention relates to a method of treating or ameliorating an auto-immune disease in a mammal, preferably a human being, comprising administering to a mammal in need thereof a therapeutically effective amount of an agent that inhibits interferon-mediated gene activation of one or more genes, for example one or more of the genes DDX58, G1P2, GAPDH, HNRPA0, IFI35, MX1, OAS3_{—}1 and RSAD2, preferably where said interferon is IFNα. Agents useful in the methods of the invention may be determined in that they reduce said activation by at least 50% in an in vitro assay at agent concentration of 10 μM or less under conditions where interferon exerts maximal activation and, preferably, wherein said reduced activation is realized for at least 3 or more of the above-recited genes. In a preferred embodiment, said agent also inhibits the activity of a molecule selected from the group consisting of histone deacetylase (HDAC), I_{K}B kinase (IKK-2), nuclear factor kB (NF-kB), ubiquitin/proteasome and Janus kinase (JAK). As recited in the methods disclosed herein, by inhibiting one of the molecules histone deacetylase (HDAC), I_{K}B kinase (IKK-2), nuclear factor kB (NF-kB), ubiquitin/proteasome and Janus kinase (JAK) is meant that an agent contemplated for use in the methods of the invention inhibits said molecule in an in vitro assay by at least 10%, preferably at least 20%, more preferably at least 30%, even more preferably at least 40%, most preferably at least 50%, when said agent is present in said assay at a concentration of 10 μM or less. Representative assays for each of the target molecules are presented in the sources cited herein demonstrating inhibitory activity of the representative agents shown in Tables 1 and 2.

In one aspect, the present invention relates to a method of treating or ameliorating an inflammatory condition in a mammal, preferably a human being, comprising administering to a mammal in need thereof a therapeutically effective amount of an agent that inhibits interferon-mediated gene activation of one or more genes, for example one or more of the genes DDX58, G1P2, GAPDH, HNRPA0, IFI35, MX1, OAS3_{—}1 and RSAD2, preferably where said interferon is IFNα, more preferably wherein said activation is reduced by at least 50% and most preferably wherein said reduced activation is realized for at least 3 or more of said genes. In a preferred embodiment, said agent also inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In another aspect, the present invention relates to a method of preventing or reducing the risk of developing an auto-immune disease or inflammatory condition in a mammal, preferably a human patient, comprising administering to a mammal in need thereof a therapeutically effective amount of an agent that inhibits interferon-mediated gene activation of one or more genes, for example one or more of the genes DDX58, G1P2, GAPDH, HNRPA0, IFI35, MX1, OAS3_{—}1 and RSAD2, preferably where said interferon is IFNα, more preferably wherein said activation is reduced by at least 50% and most preferably wherein said reduced activation is realized for at least 3 or more of said genes. In a preferred embodiment, said agent also inhibits inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In another aspect, the present invention relates to a method of preventing or reducing the risk of developing an inflammatory condition in a mammal, preferably a human patient, comprising administering to a mammal in need thereof a therapeutically effective amount of an agent that inhibits interferon-mediated gene activation of one or more genes, for example one or more of the genes DDX58, G1P2, GAPDH, HNRPA0, IFI35, MX1, OAS3_{—}1 and RSAD2, preferably where said interferon is IFNα, more preferably wherein said activation is reduced by at least 50% and most preferably wherein said reduced activation is realized for at least 3 or more of said genes. In a preferred embodiment, said agent also inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In accordance with the invention, there is provided a method of treating or ameliorating an auto-immune disease or inflammatory condition in a mammal, preferably a human being, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In another aspect, the present invention relates to a method of preventing or reducing the risk of developing an auto-immune disease or inflammatory condition in a mammal, preferably a human patient, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

In accordance with the foregoing, an agent of the invention is one that inhibits HDAC, and/or one that inhibits ubiquitin/proteasome, and/or one that inhibits nuclear factor kB (NF-kB), and/or inhibits Janus kinase (JAK), and/or inhibits I_{K}B kinase (IKK-2). Preferably, said agent inhibits more than one said molecule.

By methods known in the art, it is possible to measure gene expression, for example, by measuring transcription as the rate or amount of RNA transcribed from selected genes. Such methods include, but are in no way limited to, real time quantitative polymerase chain reaction (PCR), for example, using a Perkin-Elmer 7700 sequence detection system with gene specific primer probe combinations as designed using any of several commercially available software packages, such as Primer Express software, solid support based hybridization array technology using appropriate internal controls for quantitation, including filter, bead, or microchip based arrays, solid support based hybridization arrays using, for example, chemiluminescent, fluorescent, or electrochemical reaction based detection systems. Such methods, used to determine an expression profile of the recited gene set herein, show an activated expression profile when interferon, especially IFNα, is present. The compounds of the invention act to reduce the activated expression profile by reducing activated expression of genes related to autoimmune disease, including the genes DDX58, G1P2, GAPDH, HNRPA0, IFI35, MX1, OAS3_{—}1 and RSAD2. In preferred embodiments, activation of at least 3 of said genes is reduced as a result of administering a therapeutically effective amount of an agent of the invention. In addition, said therapeutically effective amount is able to reduce said activation to at least about 50% or below that when said agent is not present.

The present invention also relates to a method of preventing or reducing the risk of developing an auto-immune disease or inflammatory condition in a mammal, preferably a human patient, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

Agents of the invention may find use in treating any auto-immune disease, preferably those related to IFα. Among autoimmune diseases that may be treated with agents of the invention are acute disseminated encephalomyelitis (ADEM), Addison's disease, Ankylosing spondylitisis, antiphospholipid antibody syndrome, aplastic anemia, autoimmune hepatitis, autoimmune oophoritis, coeliac disease, Crohn's disease, Type I diabetes, Goodpasture's Syndrome, Graves' disease, Guillain-Barré Syndrome, Hashimoto's Disease, Idiopathic thrombocytopenic purpura, Kawasaki's disease, Systemic lupus erythematosus, multiple sclerosis, myasthenia gravis, Opsoclonus myoclonus syndrome, Optic neuritis, Ord's thyroiditis, Pemphigus, pernicious anemia, polyarthritis, rheumatoid arthritis, Reiter's syndrome, Sjögren's syndrome, Takayasu's arteritis, and Wegener's granulomatosis.

Preferably the autoimmune disease or inflammatory condition to be treated, ameliorated or prevented by a compound disclosed for use in the methods of the invention is one or more of type I diabetes, rheumatoid arthritis, psoriasis, psoriatic arthritis, ankylosing spondylitis, scleroderma, Reiter's Syndrome, systemic lupus erythematosus, dermatomyositis, Sjogren's syndrome, lupus erythematosus, multiple sclerosis, myasthenia gravis, asthma, encephilitis, inflammatory bowel disease, chronic obstructive pulmonary disease (COPD), arthritis, idiopathic inflammatory myopathies (IIM), dermatomyositis (DM, polymyositis (PM), includion body myositis or an allergic disorder.

Non-limiting representative examples of the agents useful in the methods of the invention, there are provided chemical agents having the structure of one of compounds of Tables 1 and 2.

All said compounds of Tables 1 and 2 are available from Calbiochem, Inc.

TABLE 1
Representative compounds for use in the invention

| Compound Number | Structure |
|---|---|
| 1 | 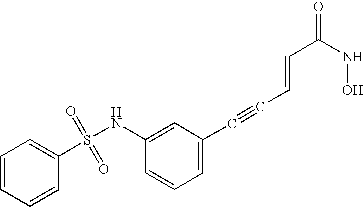 |
| 2 | 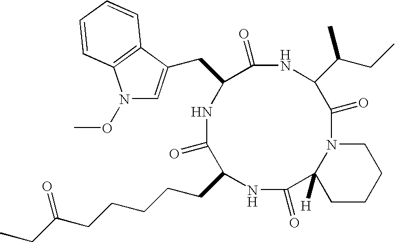 |
| 3 | 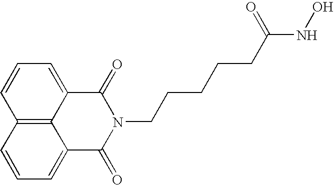 |
| 4 | 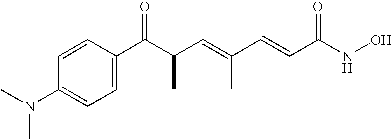 |
| 5 | 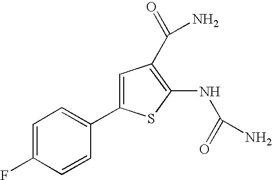 |
| 6 | 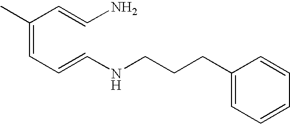 |
| 7 | 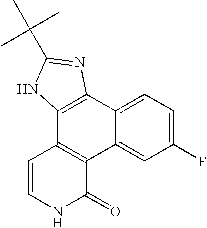 |
| 8 | 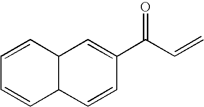 |
| 9 | 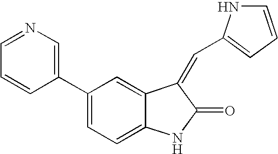 |
| 10 | 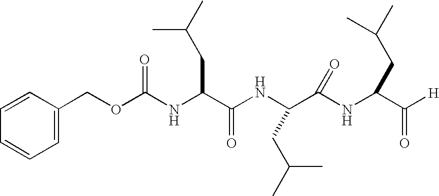 |
| 11 | 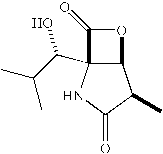 |

Inhibitory activity of these compounds with respect to the indicated target molecule has been demonstrated as follows: Compound 1 (Oxamflatin, see: Lavoie R, Bouchain G, Frechette S, Woo S H, Abou-Khalil E, Leit S, Fournel M, Yan P T, Trachy-Bourget M C, Beaulieu C, Li Z, Besterman J, Delorme D., Design and synthesis of a novel class of histone deacetylase inhibitors. Bioorg Med Chem Lett. 2001 Nov. 5; 11(21):2847-50), Compound 2 (Apicidin, see: Colletti S L, Myers R W, Darkin-Rattray S J, Gurnett A M, Dulski P M, Galuska S, Allocco J J, Ayer M B, Li C, Lim J, Crumley T M, Cannova C, Schmatz D M, Wyvratt M J, Fisher M H, Meinke P T. Broad spectrum antiprotozoal agents that inhibit histone deacetylase: structure-activity relationships of apicidin. Part 1. Bioorg Med Chem Lett. 2001 Jan. 22; 11(2):107-11), Compound 3 (scriptaid, see: Su G H, Sohn T A, Ryu B, Kern S E. A novel histone deacetylase inhibitor identified by high-throughput transcriptional screening of a compound library. Cancer Res. 2000 Jun. 15; 60(12):3137-42), Compound 4 (Trichostatin A, see: Hoshikawa Y, Kwon H J, Yoshida M, Horinouchi S, Beppu T. Trichostatin A induces morphological changes and gelsolin expression by inhibiting histone deacetylase in human carcinoma cell lines. Exp Cell Res. 1994 September; 214(1):189-97), Compound 5 (IKK-2 Inhibitor IV, see: Burke J R, Pattoli M A, Gregor K R, Brassil P J, MacMaster J F, McIntyre K W, Yang X, Iotzova V S, Clarke W, Strnad J, Qiu Y, Zusi F C. BMS-345541 is a highly selective inhibitor of I kappa B kinase that binds at an allosteric site of the enzyme and blocks NF-kappa B-dependent transcription in mice. J Biol Chem. 2003 Jan. 17; 278(3):1450-6. Epub 2002 October 25), Compound 6 (NF-kB Activation Inhibitor II, JSH-23, see: Shin H M, Kim M H, Kim B H, Jung S H, Kim Y S, Park H J, Hong J T, Min K R, Kim Y. Inhibitory action of novel aromatic diamine compound on lipopolysaccharide-induced nuclear translocation of NF-kappaB without affecting IkappaB degradation. FEBS Lett. 2004 Jul. 30; 571(1-3):50-4), Compound 7 (JAK Inhibitor I, see: Thompson J E, Cubbon R M, Cummings R T, Wicker L S, Frankshun R, Cunningham B R, Cameron P M, Meinke P T, Liverton N, Weng Y, DeMartino J A. Photochemical preparation of a pyridone containing tetracycle: a Jak protein kinase inhibitor. Bioorg Med Chem Lett. 2002 Apr. 22; 12(8):1219-23), Compound 8 (JAK3 Inhibitor V, see: Brown G R, Bamford A M, Bowyer J, James D S, Rankine N, Tang E, Torr V, Culbert E J. Naphthyl ketones: a new class of Janus kinase 3 inhibitors. Bioorg Med Chem Lett. 2000 Mar. 20; 10(6):575-9), Compound 9 (JAK3 Inhibitor VI, see: Clark M P, George K M, Bookland R G, Chen J, Laughlin S K, Thakur K D, Lee W, Davis J R, Cabrera E J, Brugel T A, VanRens J C, Laufersweiler M J, Maier J A, Sabat M P, Golebiowski A, Easwaran V, Webster M E, De B, Zhang G. Development of new pyrrolopyrimidine-based inhibitors of Janus kinase 3 (JAK3). Bioorg Med Chem Lett. 2007 Mar. 1; 17(5):1250-3), Compound 10 (MG-132, see: Rock K L, Gramm C, Rothstein L, Clark K, Stein R, Dick L, Hwang D, Goldberg A L. Inhibitors of the proteasome block the degradation of most cell proteins and the generation of peptides presented on MHC class I molecules. Cell. 1994 Sep. 9; 78(5):761-71), Compound 11 (Omuralide, see: Corey E J, Li W D. Total synthesis and biological activity of lactacystin, omuralide and analogs. Chem Pharm Bull (Tokyo). 1999 January; 47(1):1-10).

TABLE 2
Compounds for use in the invention

| Compound No. | Structure |
|---|---|
| 1 | 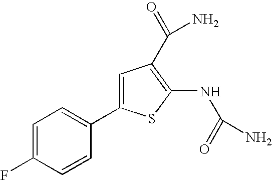 |
| 2 | 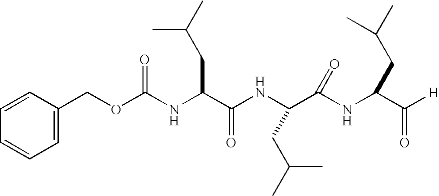 |
| 3 | 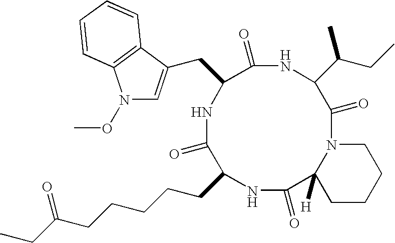 |
| 4 | 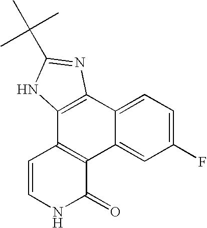 |
| 5 | 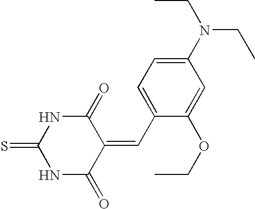 |
| 6 | 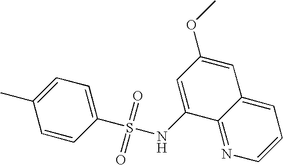 |
| 7 | 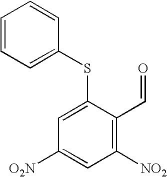 |
| 8 | 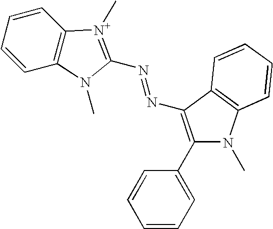 |
| 9 | 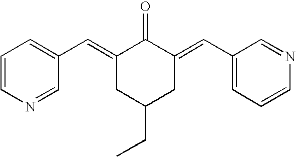 |
| 10 | 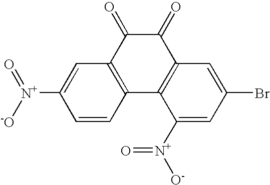 |
| 11 | 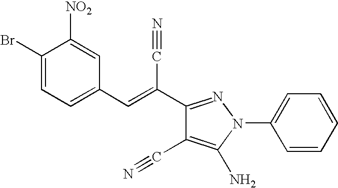 |
| 12 | 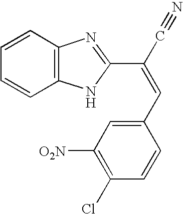 |
| 13 | 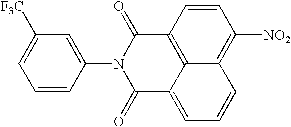 |
| 14 | 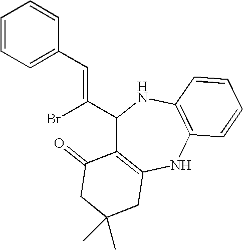 |
| 15 | 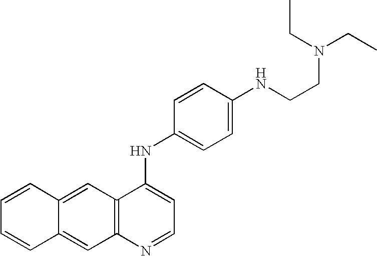 |
| 16 | 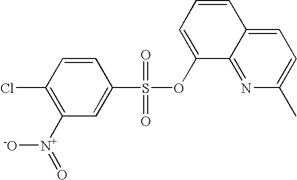 |
| 17 | 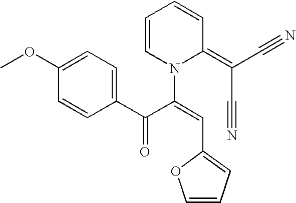 |
| 18 | 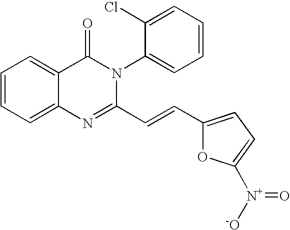 |
| 19 | 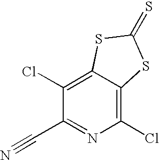 |
| 20 | 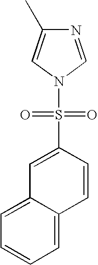 |
| 21 | 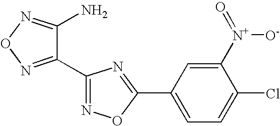 |
| 22 | 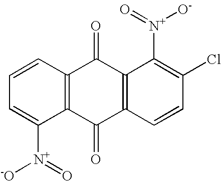 |
| 23 | 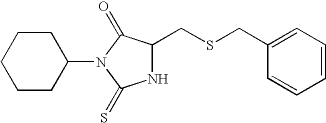 |
| 24 | 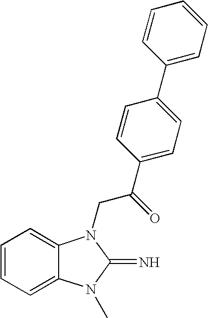 |
| 25 | 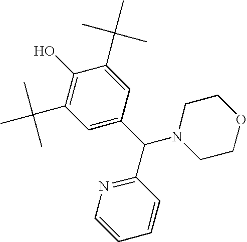 |
| 26 | 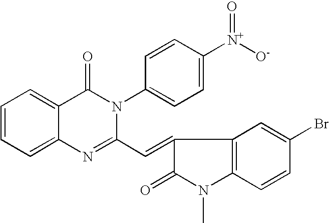 |
| 27 | 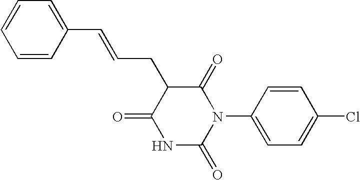 |
| 28 | 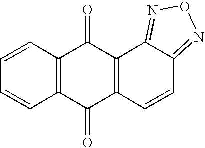 |
| 29 | 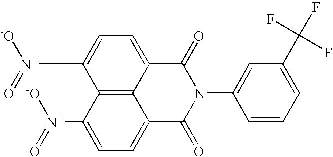 |
| 30 | 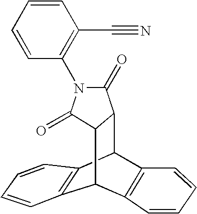 |
| 31 | 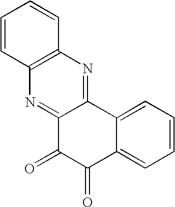 |
| 32 | 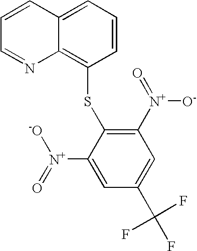 |
| 33 | 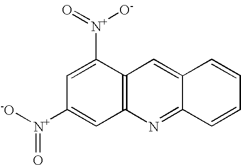 |
| 34 | 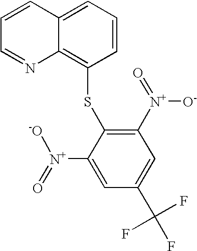 |
| 35 | 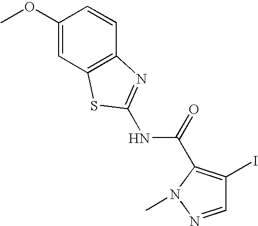 |
| 36 | 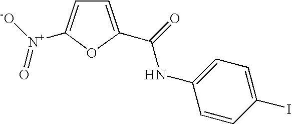 |
| 37 | 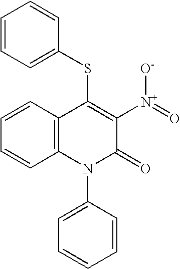 |
| 38 | 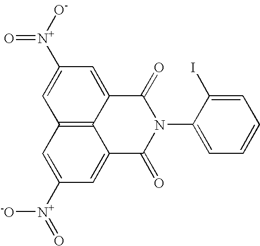 |
| 39 | 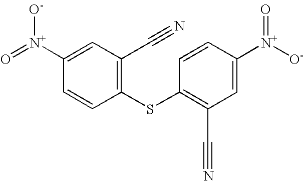 |
| 40 | 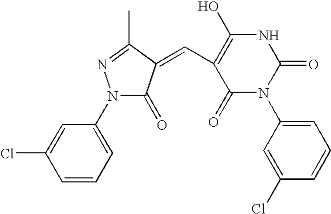 |
| 41 | 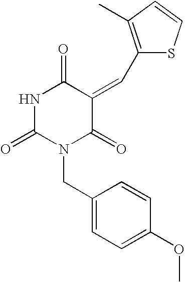 |
| 42 | 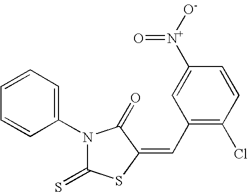 |
| 43 | 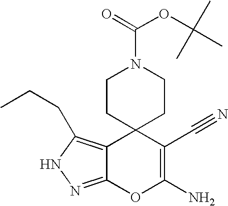 |
| 44 | 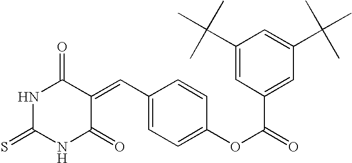 |
| 45 | 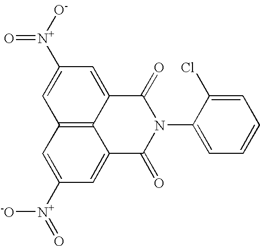 |
| 46 | 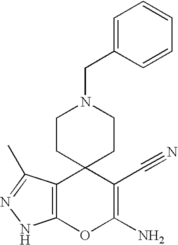 |

Inhibitory activity of compounds of Table 2 with respect to a selected target molecule as recited herein can be demonstrated by use of the assays recited above for the compounds of Table 1.

The compounds listed in Table 2 have not been reported to have interferon 2 alpha related anti-inflammatory activity. These compounds modulate interferon biological effects, preferably inhibiting said effects, more preferably not inhibiting anti-viral effects. This invention relies on the ability of these compounds to inhibit the increased steady state levels of three or more interferon-2α stimulated genes in THP-1 cells. More specifically concentrations of these compounds at 10 μM or lower partially or completely block the increase in gene expression for three or more RNA species when administered in conjunction with 100 international units of IFN2α.

The compounds recited herein reduce IFN-2α response to more than one of the recited genes, preferably at least 3 said genes. The recited compounds show substantially the same pattern of activity. In one embodiment, the compounds of Table 2 modulate the pathway response as indicated by changes in interferon 2α modulated genes. The compounds of Table 2 all modulate IFN-2α response as well as IFN-γ responses and may not modulate TNFα modulated genes. Preferably, the compounds used in the methods of the invention do not inhibit the anti-viral activity of interferons (for example, anti-HSV-1 activity). In one embodiment, a compound used in the invention inhibits such activity by inhibiting the biological activity of an expressed molecule (such as a polypeptide). In another embodiment, said compound inhibits such activity by inhibiting gene expression of said expressed molecule.

In one embodiment, the compounds of Table 1 were used to identify different mechanisms of action as identified from primary screening. If a gene is inhibited by more than 50% then it receives its maximum score (all or none type of scoring). All of the scores for all of the genes are then added and if the score is greater than 50% of the maximum score then the compound is scored as a hit. Results are shown in Table 3 where compound numbers in column 1 are those of Table 1 and MOA=mechanism of action.

TABLE 3

| | | | | HTS |
|---|---|---|---|---|
| Cpd No. | Name | Target | MOA | Score |
| 1 | Oxamflatin | HDAC | HDAC Inhibitor | 1 |
| 2 | Apicidin 1a | HDAC | HDAC Inhibitor | 0.89 |
| 3 | Scriptaid | HDAC | HDAC Inhibitor | 0.88 |
| 4 | Trichostatin A | HDAC | HDAC Inhibitor | 1 |
| 5 | IKK-2 Inhibitor IV | IKK-2 | NF-kB Pathway | 0.95 |
| 6 | NF-kB Activation | NF-kB | NF-kB Pathway | 0.94 |
| | Inhibitor II | | | |
| 7 | JAK Inhibitor I | JAK | Kinase Inhibitor | 0.9 |
| 8 | JAK3 Inhibitor V | JAK3 | Kinase Inhibitor | 0.79 |
| 9 | JAK3 Inhibitor VI | JAK3 | Kinase Inhibitor | 1 |

The compounds disclosed herein, preferably those of Tables 1 and/or 2, may be administered as stand alone formulations or may be present as part of a combination treatment along with other agents. Thus, a treatment regimen may include administering one or more of said agents together or separately, such as where 2 or more such agents are part of the same or separate compositions. In addition, other agents may be administered along with the agents of the invention.

Stimulation of healthy donor PBMCs with serum isolated from SLE patients induces the up-regulation of IFN-α pathway-associated genes (Interfon-induced genes, IFIGs), such as MX1³¹. Furthermore, expression of IFIG correlates with disease severity and organ involvement^{31, 32}. To further evaluate the role of small molecular inhibitors on the type I IFN gene signature, freshly isolated monocytes stimulated with 50% lupus serum were used in HITS assays. As shown in FIG. 4, Apicidin 1a, IKK2 inhibitor IV, and JAK inhibitors I blocked the upregulation of the six most robustly induced IFN-α signature gene set in a dose dependent manner. Apicidin 1a, IKK-2 inhibitor IV and JAK inhibitor I showed 80%, 77% and 60% inhibition respectively, at a concentration (1 μm) that did not exhibit cytotoxicity. Importantly, these experiments were performed with SLE serum from patients with well characterized biochemical profiles. The acquired data suggest that JAK inhibitor I, IKK-2 inhibitor IV, and Apicidin 1a are effective inhibitors of the IFN-α gene signature induced by SLE serum. Since the biological activity of SLE serum has been associated with pathogenesis, our results suggest for the first time that small molecule inhibitors targeting HDAC, NF-κb and Jak/Stat signaling pathways could modulate SLE disease activity.

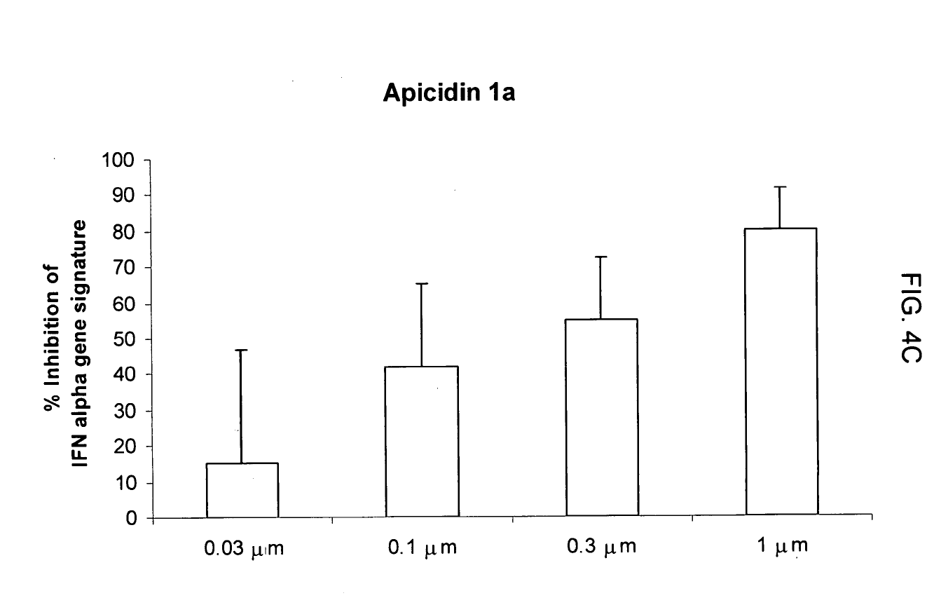

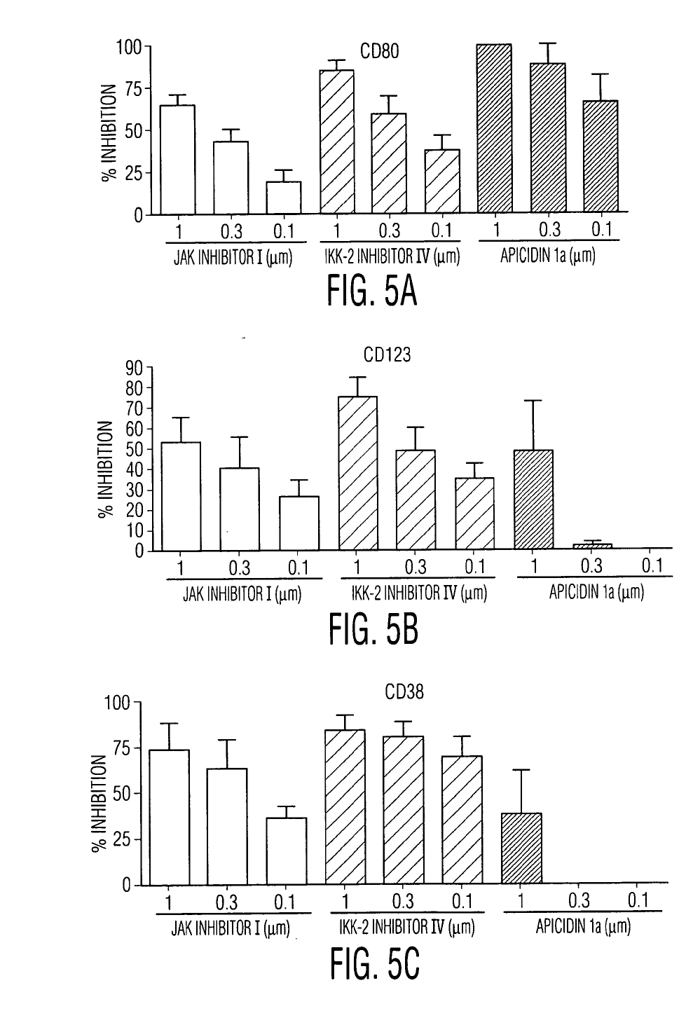

Since deregulated differentiation of DCs induced by IFN-α may be a critical contributing factor in SLE¹³, the impact of Apicidin 1a, IKK2 inhibitor IV, and JAK inhibitors in DC differentiation was examined. Human monocytes were stimulated with IFN-α and the expression of CD38 CD80 and CD123 differentiation markers was monitored³³. Treatment of Jak inhibitor I and IKK-2 inhibitor IV, results in a dose-dependent inhibition of CD38 CD80 and CD123 expression (FIG. 5). Apicidin 1a blocks CD80 expression however, the inhibition of CD38 and CD123 expression is limited to the highest dose of 1 μM. In summary, the results indicate that JAK/Stat and NF-κb signaling pathways are critical mediators of IFN-α-induced CD38, CD80, and CD123 expression in differentiating monocytes.

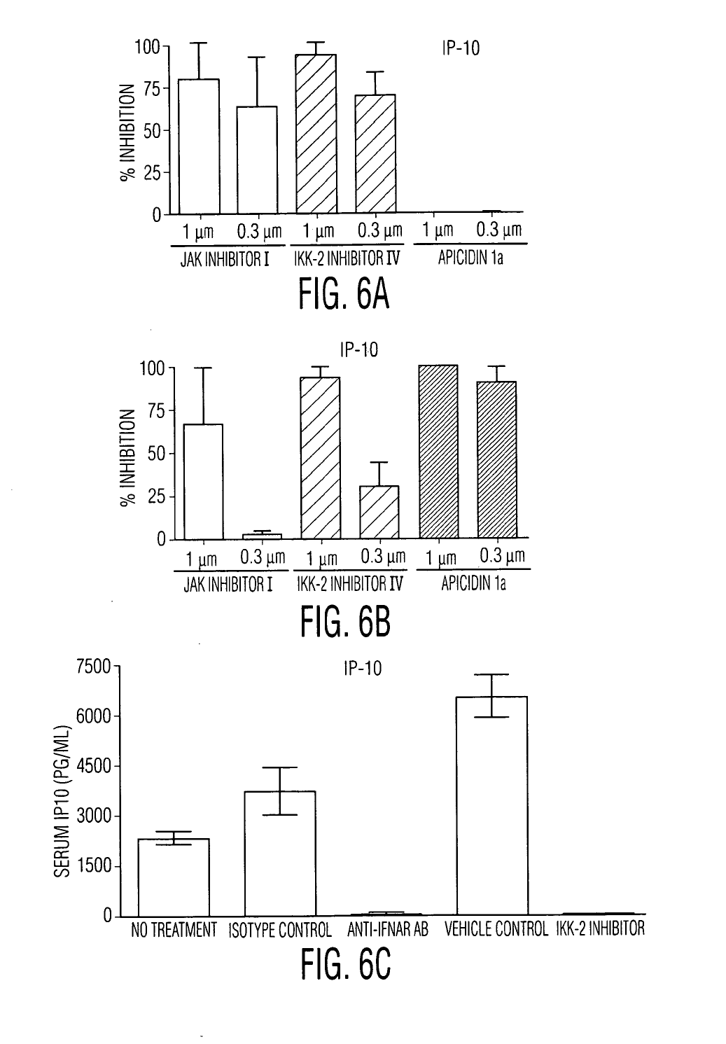

Multiple chemokines, including monocyte chemo-attractant protein-1 (MCP-1) and activated T cell chemokine interferon inducible protein 10 (IP-10) regulate leukocytes migration and infiltration into inflammated organs³⁴. Expression of MCP-1 and IP-10 are elevated in serum of SLE patients^{34, 35} and in monocytes of healthy donors stimulated in vitro by IFN-α³⁶. Consequently, the effect of Apicidin 1a, IKK2 inhibitor IV, and JAK inhibitors in IP-10 and MCP-1 expression induced by IFN-α from human monocytes was examined. As indicated in FIGS. 6A and 6B, Jak inhibitor I and IKK-2 inhibitor IV block the expression of IP-10 induced by IFN-α. On the other hand, the effect on MCP-1 expression is restricted to the highest dose of Jak inhibitor I and IKK-2 inhibitor IV. Apicidin 1a treatment totally neutralizes MCP-1 expression, but in contrast no neutralization of IP-10 expression is observed.

Next, the effect of IKK-2 inhibitor IV in IP-10 and MCP-1 expression was examined in vivo. To induce IP-10 and MCP-1 expression, mice were infected with adenovirus encoding IFN-α5. As a result, IP-10 but not MCP-1 levels were detected in serum (data not shown). Treatment of IKK-2 inhibitor IV and a surrogate mouse anti-interferon receptor antibody (5A3), used as positive control, resulted in 98% inhibition of levels of serum IP-10. These observations illustrate the robustness of our strategy for identifying small molecule inhibitors with desirable immunosuppressive effect.

Herpes Simplex Virus-1 (HSV-1) represents one of the major recurrent virus infections observed in SLE patients³⁷. Type-I and Type-II IFN signals are known to block HSV-1 dissemination in mice³⁸, and as a consequence, a therapeutic approach that neutralizes their combined activity may constitute an important safety concern. Therefore, the impact of Apicidin 1a, IKK2 inhibitor IV, and JAK inhibitors on HSV-1 replication regulated by IFN-α in Hep-2 cells was examined in vitro. HSV-1/luciferase was used to infect Hep-2 cells, and viral replication was monitored by luciferase expression. We first confirmed that reporter gene activity rose concomitantly and proportionally with the detection of viral progeny (data not shown).

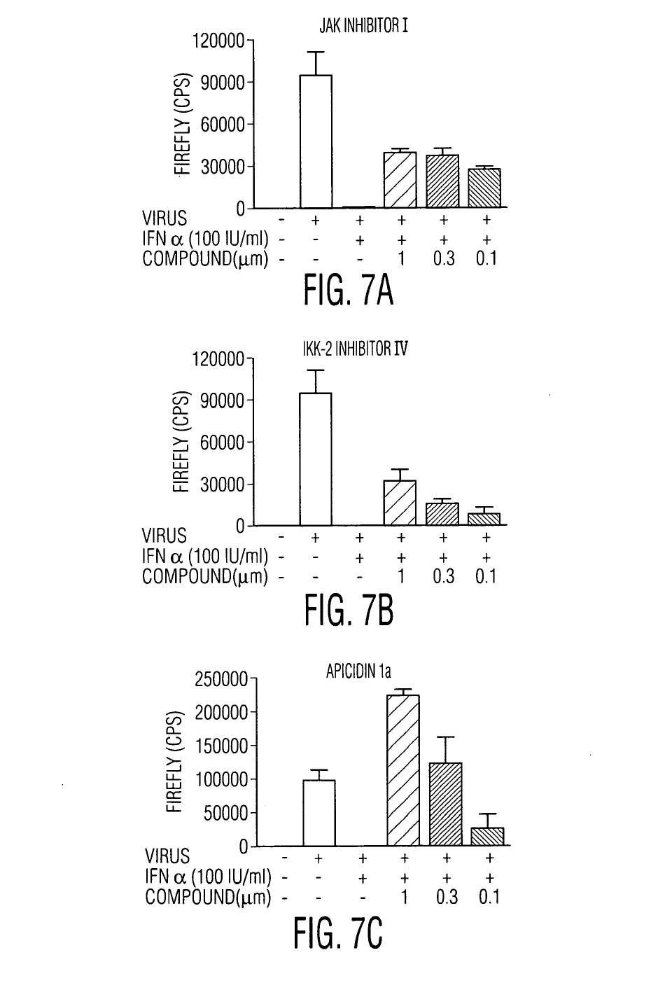

As shown in FIG. 7, in absence of IFN-α, luciferase expression indicates high levels of HSV-1 replication. IFN-α treatment significantly reduced viral replication. Both the Jak1 and IKK2-inhibitor IV retained the majority of IFN-α dependent anti-viral activity (FIGS. 7A and 7B), even at higher doses that inhibited IFN gene signatures, monocyte activation and chemokine production. In contrast, Apicidin 1a inhibited anti-viral effects of IFN-α at low dose (0.1 μm), but at higher dose where this drug affectively inhibited MCP-1 production, IFN-α dependent anti-viral activity was abolished or viral growth was actually promoted (FIG. 7C). Based on these results, both Jak I and IKK-2 IV inhibitors emerge as possible lead candidates with desirable immunosuppressive and dissociated anti-viral effects.

For example, the agents useful in the methods of the invention may be administered together with one or more additional therapeutic agents, especially those having an anti-inflammatory effect. Such additional anti-inflammatory agent may be a steroid or a non-steroid, wherein said steroid is preferably a corticosteroid, preferably one or more of cortisone, hydrocortisone, prednisone, prednisolone, methylprednisolone, triamcinolone, betamethasone, beclamethasone and dexamethasone.

For administration purposes, an effective amount of autoimmune inhibitor is expected to vary from about 1 milligram per kilogram of body weight per day (mg/kg/day) to about 500 mg/kg/day. Preferred amounts are expected to vary from about 10 to about 100 mg/kg/day, more preferably from about 20 to about 100 mg/kg/day, even more preferably from about 50 to about 100 mg/kg/day and most preferably between about 60 to about 90 mg/kg/day. Treatment regimens may call for single doses at intervals or may involve repeated doses, such as every day for 3 or more days. It is also within the invention to administer a test dose of one or more of the agents of the invention and assess the effect on a patient, such as one in need thereof, before administering further doses.

An agent for use in any of the methods of the invention can be administered in any form or mode that makes the agent available in effective amounts, including either oral and parenteral routes. These can include any one or more of administering orally, subcutaneously, intramuscularly, intravenously, transdermally, intranasally, and rectally. Administration by more than one such route of an agent of the invention or administration of an agent of the invention and another agent of the invention by a different route is not precluded. In addition, where additional therapeutics are to be administered along with an agent as disclose herein, these may each be administered by a different route and still be within the methods of the invention. Thus, for example, where an agent of the invention is administered orally and a steroid is administered intravenously, said regimen still falls within the methods of the invention.

The chemical compounds disclosed herein for use in the methods of the invention may be administered alone or as a pharmaceutical composition in combination with pharmaceutically acceptable carriers, the proportion and nature of which are determined by the solubility and chemical properties of the compound selected, the selected route of administration, and standard pharmaceutical practice in this field. The compounds of the invention, while effective themselves, may be formulated and administered in the form of a salt, acid, base, prodrug or metabolite of any of the structures disclosed for compounds 1 through 46 above. The only requirement for such drug form is that it is non-toxic to the recipient and effective for the desired therapeutic result (i.e., effective and bioavailable). Such different forms of the compounds useful herein may be selected based on their solubility in the pharmaceutical carrier used for administration. Such dosage forms may be in the form of tablets, powders, granules, capsules, suppositories, solution, suspensions, and the like. The compounds useful in the methods of the invention may also be enclosed in gelatin capsules or compressed into tablets. For oral administration, the compounds may be incorporated with carriers and used in the form of tablets, troches, capsules, elixirs, suspensions, syrups, wafers, chewing gums and the like, wherein one or more of compounds 1 through 46 represent the active ingredients.

The carriers useful for such preparations are any of those well known in the art. In certain embodiments a desirable route of administration can be by pulmonary aerosol. Techniques for preparing aerosol delivery systems containing polypeptides are well known to those of skill in the art. (see, for example, Sciarra and Cutie, “Aerosols,” in Remington's Pharmaceutical Sciences 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates polycaprolactones, polyesteramides, polyorthoesters, poiyhydroxybutyric acids, such as poly-D-(−)-3-hydroxybutyric acid (European Patent No. 133,988), copolymers of L-glutamic acid and gamma-ethyl-L-glutamate (Sidman, K R. et at, Biopolymers 22: 547-556), poly(2-hydroxyethyl methacrylate) or ethylene vinyl acetate (Langer, ft et at, J. Biomed. Mater. Res. 15:267-277; Langer, B. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono-, di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fined implants; and the like. Specific examples include, but are not limited to: (a) erosional systems in which the agent is contained in a form within a matrix such as those described in 13.5. U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) maybe present in any concentration sufficient to modulate the osmotic properties of the formulation.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro cclluioses, polymers of acrylic and methaerylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, and other prolamines and hydrophobic proteins, copolymers and mixtures thereof. In general, these materials degrade either by enzymatic hydrolysis or exposure to water in vivo, by surface or bulk erosion.

### Example 1

### Gene Expression Analysis

Human U133A microarrays (Affymetrix Inc. Santa Clara, Calif.) were used to profile transcriptional changes in THP-1 cells stimulated with cytokines. THP-1 cells seeded at 5×10⁵ cells/ml were treated with 100 IU/ml IFN-α, 10 ng/ml IFN-α, 10 ng/ml TNF-α, or vehicle control for four hours. Total RNA was isolated in Trizol reagent (Invitrogen Inc, Carlsbad, Calif.) and purified on RNeasy plate (Qiagen Inc, Valencia, Calif.). The purified total RNA from eight biological replicates for IFN-α and IFN-γ treatments, six replicates of TNFα treatments, and 14 replicates of vehicle only controls were processed and hybridized on HT_HG-U133A high-throughput 96 well array plates according to Affymetrix High-Throughput Array platform protocols provided by the microarray supplier. The raw data files were processed and normalized with RMA express. All the stimulated gene expression data sets were normalized to the vehicle control treatments for the pathway gene marker set analysis. Raw data are not shown.

The specific IFN-α pathway gene signature set was identified using genome-wide expression analysis and normalizing to vehicle only controls.

We selected genes showing either up-regulation or repression after IFN-α stimulation by using the Significance Analysis for Microarray (SAM) package⁴⁹. Genes with FDR <0.01 and showing more than a 1.4-fold change in expression level were selected as activation markers. The selected genes then were ranked using a signal-to-noise (SNR) statistical approach⁴³. To improve the specificity of the gene signature, we further removed any gene from the IFN-α pathway marker set that showed significant modulation in response to IFN-γ or TNF-α activation. We selected six genes (MX1, OAS3, DDX58, RSAD2, G1P2, and IFI35) that were upregulated at least 2-fold and one down-regulated gene (HNRPA0) as the IFN-α signature gene set for our High-Throughput Integrated Transcriptional Screening (HITS)

THP-1 cells seeded at 8×10⁵ cells/ml were treated with either compound (10 μM final concentration) or vehicle control (0.1% DMSO) for 30 minutes prior to a four hour stimulation with 100 IU/ml IFN-α or PBS. Plates were incubated at 37° C. in a humidified incubator. Cell lysis and RNA isolation were carried out according to the manufacturer's instructions (TurboCapture mRNA Kit, Qiagen Inc.). PCR primer pairs were designed for each of the genes identified through the global gene expression analysis using Primer 3 based method (see below for primer sequences). Each primer was evaluated for the efficient production of a single band of appropriate size in the presence of template RNA and the lack of distinct amplification products in a non-template control. Real time PCR reactions were carried out in SYBR green master mix (Quanta biosciences) on ABI9700 thermocyclers.

Oligo pairs used for RT-PCR for determination of gene expression increase or decrease are shown in Table 4:

TABLE 4

| | | |
|---|---|---|
| Gene | Forward Primer | KReverse Primer |

| | | |
|---|---|---|
| IFI35 | ggagtggctcagcgtctgt | actggctgcgacctgatct |
| | (SEQ ID NO: 1) | (SEQ ID NO: 2) |
| OAS3 | tgaaggctgctgtgtgaagt | cacacacacatgtacacaatctctc |
| | (SEQ ID NO: 3) | (SEQ ID NO: 4) |
| G1P2 | cgaactcatctttgccagtaca | gagctgctcagggacacc |
| | (SEQ ID NO: 5) | (SEQ ID NO: 6) |
| RSAD2 | tggatagcaaatcctgagacaat | cctgtgtgtattccttctttttagc |
| | (SEQ ID NO: 7) | (SEQ ID NO: 8) |
| HNRPA0 | gggtgggttcagagtacctttt | gcttcttagtatagctttgagccttc |
| | (SEQ ID NO: 9) | (SEQ ID NO: 10) |
| DDX58 | tgaactgtaagggttagtggagagt | aaataatccatttgtattgggtct |
| | (SEQ ID NO: 11) | (SEQ ID NO: 12) |
| MX1 | ggacatcactgctctcatgc | tttatggccttcttgaaaattg |
| | (SEQ ID NO: 13) | (SEQ ID NO: 14) |

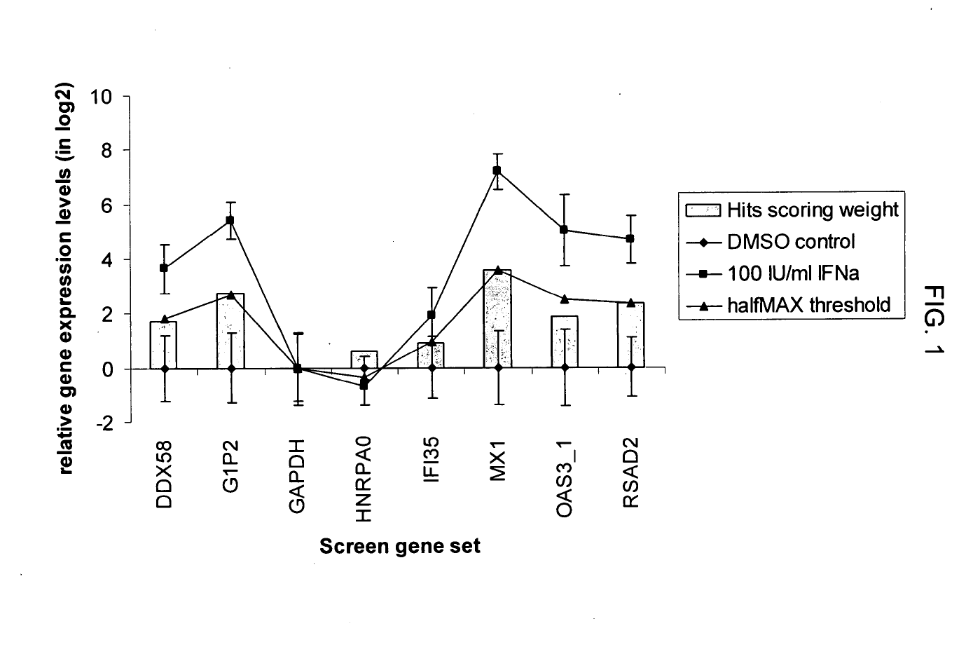

We used the house keeping gene, GAPDH, as the normalization control for the IFN-α gene signature set to correct the overall variability in the qPCR based HITS process. The corrected profiles were then normalized to the basal gene expression levels determined by using the vehicle only treatments. We subtracted the vehicle only control gene value from the test compound gene profiles, gene per gene (see FIG. 1).

### Example 2

### Dose Dependent Gene Transcriptional Inhibition (TI)

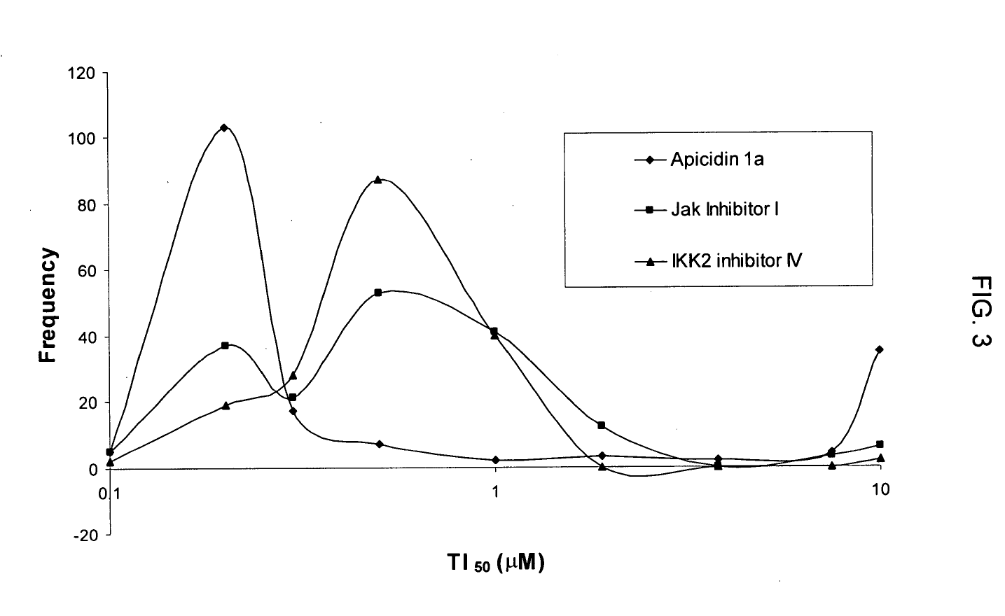

2.4×10⁴ THP-1 cells were seeded in 384 well culture plate in 30 μl culture medium. Test compounds in 100% DMSO were diluted and yielded final concentrations from 10 μM to 5 nM in the cell culture directly. Cells were treated with test compounds together with 100 IU/ml IFN-α for 4 hours before they were lysed for RNA isolation. The IFN-α markers were profiled with the purified RNA using the HITS assay. The IFN-α signature genes were normalized to the GAPDH control, and subsequently the median mRNA level for each of the seven genes was calculated. The dose dependent gene transcriptional inhibition curves were then generated for each of the test compounds. Results are shown in FIG. 3.

### Example 3

### Human PBMC Stimulation with IFN-α or Patient Serum

SLE patient and control serum were purchased from Bioreclamation (Hicksville, N.Y.). IFN-α 2a was obtained from PBL Biomedical laboratories (Piscataway, N.J.). Fresh PBMCs from healthy donor were prepared by Ficoll-hypaque fraction according to the manufactory's instruction. Cells were cultured at 2×10⁵ cells/0.1 ml in 96-well flat-bottomed plates in culture medium.

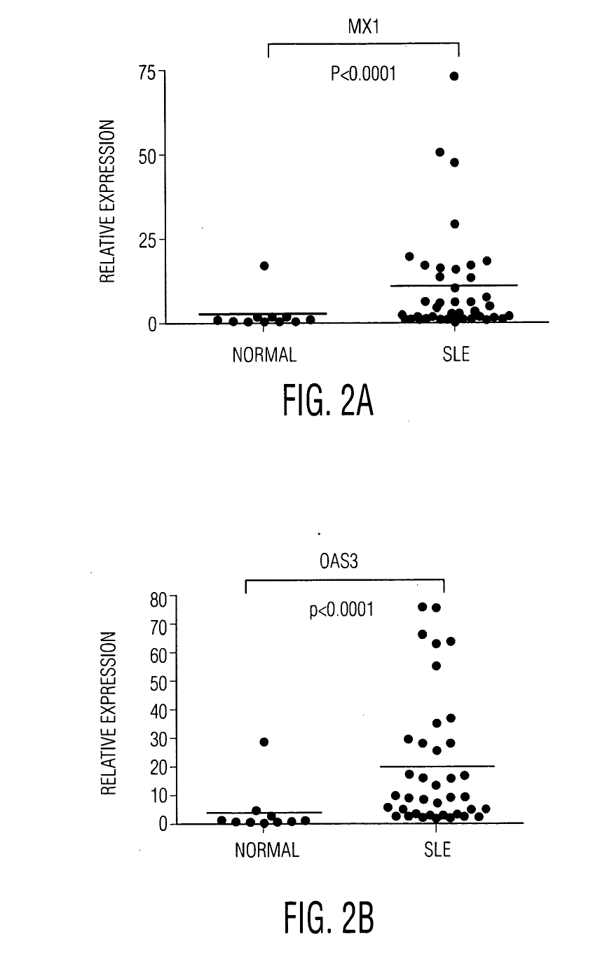

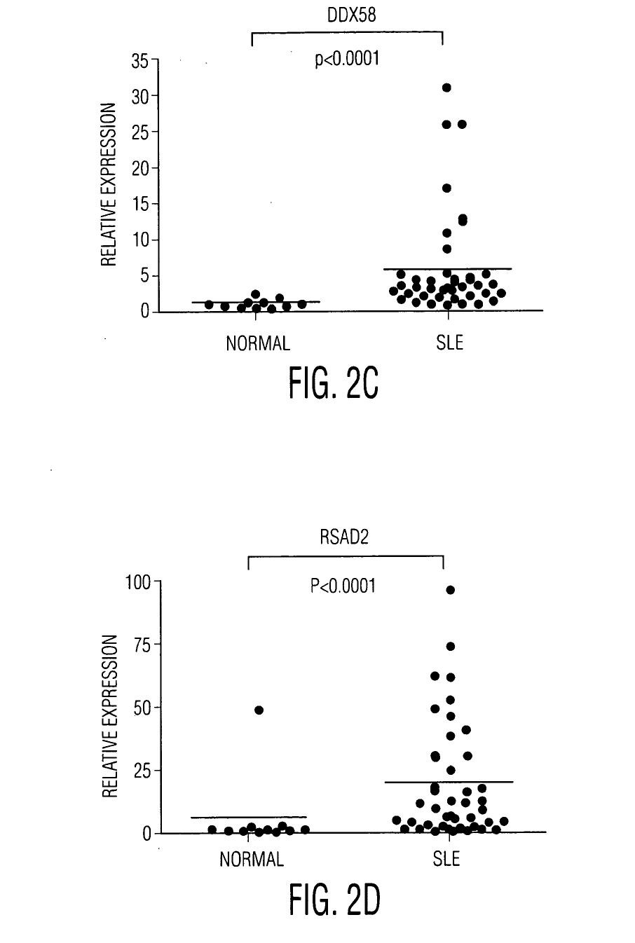

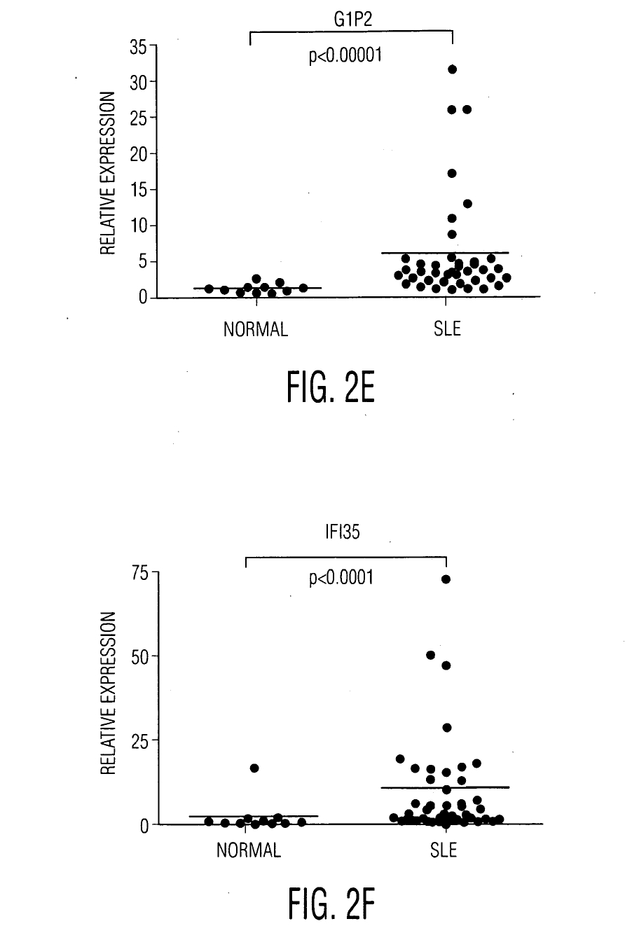

To determine the effect of compounds on gene expression, compounds and vehicle controls were pre-incubated with cells for 30 min at 37° C. before stimulation with 50% lupus serum or 100 IU/ml IFN-α 2a were incubated with PBMC. After 6 h stimulation, cells were lysed in Qiagen 2XTCL lysis buffer followed by HITS analysis (see FIG. 2).

### Example 4

### Cell Surface Marker Expression and Cytokine Production

For monocyte differentiation assays, human monocytes were isolated from the blood of healthy donors according to manufactory's instructions (Miltenyi Biotec Inc; Auburn Calif.). 5×10⁵ freshly prepared monocytes were pre-incubated with compounds or vehicle control for 30 min. Cells were then incubated in RPMI 1640 medium (Invitrogen, Carlsbad, Calif.) including 10% fetal calf serum (Invitrogen) supplemented with 500 IU/ml IFN-α 2a for 48 hr. Cell surface protein were stained with FITC-labeled anti-CD80, PE-labeled anti-CD123, PEcy7 labeled anti-CD38, APC cy7 labeled MHC class II (all BD, San Jose, Calif.) and analyzed on a FACS Calibur flow cytometer (BD). Cytokine and chemokine levels in supernatants from monocyte differentiation assays were measured using searchlight human cytokine array (Pierce Inc, Woburn, Mass.). (see FIG. 5)

### Example 5

### In Vitro Anti-Viral Assay

Hep-2 cells were propagated in MEM (Invitrogen) with 10% fetal bovine serum. HSV-1 recombination virus with both firefly and Renilla luciferase genes in a divergent orientation from a single multiple cloning site were a gift from Dr. Leib's lab. Hep-2 cells were seeded at 2×10⁴ cells/well in 96 well plate, 24 h post seeding, cells were infected with virus at an MOI of 5, after absorption for 1 h at 37° C., free viral particles were removed by aspiration, cells were washed, and 200 μl medium containing IFN-α with or without test compounds. After 48 h incubation, cells were washed and lysed in 20 μl of Passive Lysis Buffer (Promega Inc; Madison, Wis.) frozen, thawed, and assayed for firefly and Renilla luciferase activity using the Dual Luciferase Assay kit (Promega). (See FIG. 7)

### Example 6

### IFN-α Induced Chemokine Release In Vivo

Female NZBW/F1 mice (Jackson Laboratories, Bar Harbor, Me.) were housed in pathogen-free conditions with access to food and water. At 11 weeks of age, mice were randomized and placed into control (n=4-5) and treatment groups (n=6). TPCA-1 was administered in 0.9% DMSO, 7% dimethylacetoacetamide (Aldrich Chemical Co., Milwaukee, Wis.) and 10% Cremophor EL (Sigma-Aldrich) 2 hours prior to stimulation. Anti-mouse IFN-α receptor antibodies (5A3) or mouse IgG1 isotype control antibodies were administered 16 hours prior to stimulation as a positive control. Mice were stimulated by adenovirus delivery of IFN-α5, 1×10¹⁰ viral particles were injected intravenously. Six hours post-stimulation, the mice were sacrificed by CO₂ exposure and blood samples were collected by cardiac puncture. Serum levels of IP10 were assessed by ELISA (R&D Systems, Minneapolis, Minn.). (See FIG. 6)

### REFERENCE LIST

1. Boumpas, D. T. et al. Systemic lupus erythematosus: emerging concepts. Part 2: Dermatologic and joint disease, the antiphospholipid antibody syndrome, pregnancy and hormonal therapy, morbidity and mortality, and pathogenesis. Ann Intern Med 123, 42-53 (1995).
2. Bengtsson, A. A. et al. Activation of type I interferon system in systemic lupus erythematosus correlates with disease activity but not with antiretroviral antibodies. Lupus 9, 664-671 (2000).
3. Ronnblom, L. & Alm, G. V. Systemic lupus erythematosus and the type I interferon system. Arthritis Res Ther 5, 68-75 (2003).
4. Pascual, V., Farkas, L., & Banchereau, J. Systemic lupus erythematosus: all roads lead to type I interferons. Curr Opin Immunol 18, 676-682 (2006).
5. Nordmark, G., Alm, G. V., & Ronnblom, L. Mechanisms of Disease: primary Sjogren's syndrome and the type I interferon system. Nat Clin Pract Rheumatol 2, 262-269 (2006).
6. Pestka, S., Langer, J. A., Zoon, K. C., & Samuel, C. E. Interferons and their actions. Annu Rev Biochem 56, 727-777 (1987).
7. Pestka, S., Krause, C. D., & Walter, M. R. Interferons, interferon-like cytokines, and their receptors. Immunol Rev 202, 8-32 (2004).
8. Stark, G. R., Kerr, I. M., Williams, B. R., Silverman, R. H., & Schreiber, R. D. How cells respond to interferons. Annu Rev Biochem 67, 227-264 (1998).
9. Darnell, J. E., Jr., Kerr, I. M., & Stark, G. R. Jak-STAT pathways and transcriptional activation in response to IFNs and other extracellular signaling proteins. Science 264, 1415-1421 (1994).
10. Platanias, L. C. Mechanisms of type-I- and type-II-interferon-mediated signalling. Nat Rev Immunol 5, 375-386 (2005).
11. Ito, T., Wang, Y. H., & Liu, Y. J. Plasmacytoid dendritic cell precursors/type I interferon-producing cells sense viral infection by Toll-like receptor (TLR) 7 and TLR9. Springer Semin Immunopathol 26, 221-229 (2005).
12. Ozato, K., Tsujimura, H., & Tamura, T. Toll-like receptor signaling and regulation of cytokine gene expression in the immune system. Biotechniques Suppl, 66-8, 70, 72 (2002).
13. Blanco, P., Palucka, A. K., Gill, M., Pascual, V., & Banchereau, J. Induction of dendritic cell differentiation by IFN-alpha in systemic lupus erythematosus. Science 294, 1540-1543 (2001).
14. Crow, M. K., Kirou, K. A., & Wohlgemuth, J. Microarray analysis of interferon-regulated genes in SLE. Autoimmunity 36, 481-490 (2003).
15. Ytterberg, S. R. & Schnitzer, T. J. Serum interferon levels in patients with systemic lupus erythematosus. Arthritis Rheum 25, 401-406 (1982).
16. Kirou, K. A. et al. Coordinate overexpression of interferon-alpha-induced genes in systemic lupus erythematosus. Arthritis Rheum 50, 3958-3967 (2004).
17. Baechler, E. C., Gregersen, P. K., & Behrens, T. W. The emerging role of interferon in human systemic lupus erythematosus. Curr Opin Immunol 16, 801-807 (2004).
18. Bennett, L. et al. Interferon and granulopoiesis signatures in systemic lupus erythematosus blood. J Exp Med 197, 711-723 (2003).
19. Baechler, E. C. et al. Interferon-inducible gene expression signature in peripheral blood cells of patients with severe lupus. Proc Natl Acad Sci USA 100, 2610-2615 (2003).
20. von, W. P. et al. MX homologous protein in mononuclear cells from patients with systemic lupus erythematosus. Arthritis Rheum 32, 914-918 (1989).
21. Mathian, A., Weinberg, A., Gallegos, M., Banchereau, J., & Koutouzov, S. IFN-alpha induces early lethal lupus in preautoimmune (New Zealand Black×New Zealand White) F1 but not in BALB/c mice: J Immunol 174, 2499-2506 (2005).
22. Santiago-Raber, M. L. et al. Type-I interferon receptor deficiency reduces lupus-like disease in NZB mice. J Exp Med 197, 777-788 (2003).
23. Nestle, F. O. et al. Plasmacytoid predendritic cells initiate psoriasis through interferon-alpha production. J Exp Med 202, 135-143 (2005).
24. Dietrich, L. L., Bridges, A. J., & Albertini, M. R. Dermatomyositis after interferon alpha treatment. Med Oncol 17, 64-69 (2000).
25. Banchereau, J. & Pascual, V. Type I interferon in systemic lupus erythematosus and other autoimmune diseases. Immunity 25, 383-392 (2006).
26. Bol, D. & Ebner, R. Gene expression profiling in the discovery, optimization and development of novel drugs: one universal screening platform. Pharmacogenomics 7, 227-235 (2006).
27. Stoughton, R. B. & Friend, S. H. How molecular profiling could revolutionize drug discovery. Nat Rev Drug Discov 4, 345-350 (2005).
28. Hieronymus, H. et al., Gene expression signature-based chemical genomic prediction identifies a novel class of HSP90 pathway modulators. Cancer Cell 10, 321-330 (2006).
29. Lamb, J. et al. The Connectivity Map: using gene-expression signatures to connect small molecules, genes, and disease. Science 313, 1929-1935 (2006).
30. Wei, G. et al. Gene expression-based chemical genomics identifies rapamycin as a modulator of MCL1 and glucocorticoid resistance. Cancer Cell 10, 331-342 (2006).
31. Feng, X. et al. Association of increased interferon-inducible gene expression with disease activity and lupus nephritis in patients with systemic lupus erythematosus. Arthritis Rheum 54, 2951-2962 (2006).
32. Hua, J., Kirou, K., Lee, C., & Crow, M. K. Functional assay of type I interferon in systemic lupus erythematosus plasma and association with anti-RNA binding protein autoantibodies. Arthritis Rheum 54, 1906-1916 (2006).
33. Dall'era, M. C., Cardarelli, P. M., Preston, B. T., Witte, A., & Davis, J. C., Jr. Type I interferon correlates with serological and clinical manifestations of SLE. Ann Rheum Dis 64, 1692-1697 (2005).
34. Lit, L. C., Wong, C. K., Tam, L. S., Li, E. K., & Lam, C. W. Raised plasma concentration and ex vivo production of inflammatory chemokines in patients with systemic lupus erythematosus. Ann Rheum Dis 65, 209-215 (2006).
35. Narumi, S., Takeuchi, T., Kobayashi, Y., & Konishi, K. Serum levels of ifn-inducible PROTEIN-10 relating to the activity of systemic lupus erythematosus. Cytokine 12, 1561-1565 (2000).
36. Svane, I. M. et al. Characterization of monocyte-derived dendritic cells maturated with IFN-alpha. Scand J Immunol 63, 217-222 (2006).
37. Rider, J. R., Ollier, W. E., Lock, R. J., Brookes, S. T., & Pamphilon, D. H. Human cytomegalovirus infection and systemic lupus erythematosus. Clin Exp Rheumatol 15, 405-409 (1997).
38. Harle, P., Lauret, E., Pitha, P. M., De, M. E., & Carr, D. J. Expression of human and macaque type I IFN transgenes interferes with HSV-1 replication at the transcriptional and translational levels: IFN-beta is more potent than IFN-alpha 2. Virology 290, 237-248 (2001).
39. Vilcek, J. Fifty years of interferon research: aiming at a moving target. Immunity 25, 343-348 (2006).
40. Nguyen, K. B. of al. Critical role for STAT4 activation by type 1 interferons in the interferon-gamma response to viral infection. Science 297, 2063-2066 (2002).
41. Rothfuchs, A. G. of al. STAT1 regulates IFN-alpha beta- and IFN-gamma-dependent control of infection with Chlamydia pneumoniae by nonhemopoietic cells. J Immunol 176, 6982-6990 (2006).
42. O'Neill, L. A. Targeting signal transduction as a strategy to treat inflammatory diseases. Nat Rev Drug Discov 5, 549-563 (2006).
43. Boulares, A. H., Ferran, M. C., & Lucas-Lenard, J. NF-kappaB activation Is delayed in mouse L929 cells infected with interferon suppressing, but not inducing, vesicular stomatitis virus strains. Virology 218, 71-80 (1996).
44. Wei, L. et al. NFkappaB negatively regulates interferon-induced gene expression and anti-influenza activity. J Biol Chem 281, 11678-11684 (2006).
45. Gregory, D., Hargett, D., Holmes, D., Money, E., & Bachenheimer, S. L. Efficient replication by herpes simplex virus type 1 involves activation of the IkappaB kinase-IkappaB-p65 pathway. J Virol 78, 13582-13590 (2004).
46. Birrell, M. A. of al. Ikappa-B kinase-2 inhibitor blocks inflammation in human airway smooth muscle and a rat model of asthma. Am J Respir Crit Care Med 172, 962-971 (2005).
47. Chang, H. M. et al. Induction of interferon-stimulated gene expression and antiviral responses require protein deacetylase activity. Proc Natl Acad Sci USA 101, 9578-9583 (2004).
48. Nusinzon, I. & Horvath, C. M. Unexpected roles for deacetylation in interferon- and cytokine-induced transcription. J Interferon Cytokine Res 25, 745-748 (2005).
49. Tusher, V. G., Tibshirani, R., & Chu, G. Significance analysis of microarrays applied to the ionizing radiation response. Proc Natl Acad Sci USA 98, 5116-5121 (2001).

1. A method of treating or ameliorating an auto-immune disease or inflammatory condition in a mammal, comprising administering to a mammal in need thereof a therapeutically effective amount of an agent that inhibits the activity of a molecule selected from the group consisting of histone deacetylase (HDAC), I_{K}B kinase (IKK-2), nuclear factor kB (NF-kB), ubiquitin/proteasome and Janus kinase (JAK).

2. (canceled)

3. The method of claim 1, wherein said agent inhibits more than one of said molecules.

4-8. (canceled)

9. The method of claim 1, wherein the autoimmune disorder or inflammatory disorder is selected from the group consisting of insulin-dependent (type I) diabetes, rheumatoid arthritis, psoriasis, psoriatic arthritis, ankylosing spondylitis, scleroderma, Reiter's Syndrome, systemic lupus erythematosus, dermatomyositis, Sjogren's syndrome, lupus erythematosus, multiple sclerosis, myasthenia gravis, asthma, encephilitis, inflammatory bowel disease, chronic obstructive pulmonary disease (COPD), arthritis, idiopathic inflammatory myopathies (IIM), dermatomyositis (DM, polymyositis (PM), includion body myositis and an allergic disorder.

10. The method of claim 9, wherein said auto-immune disease is one or more of insulin-dependent (Type I) diabetes, rheumatoid arthritis, psoriasis, myasthenia gravis, multiple sclerosis, or systemic lupus erythematosus (SLE).

11. The method of claim 10, wherein said auto-immune disease is systemic lupus erythematosus (SLE).

12. The method of claim 1, wherein said agent is one or more of the compounds of Table 1 or Table 2.

13. (canceled)

14. A method of treating or ameliorating an auto-immune disease disease or inflammatory condition in a mammal, comprising administering to a mammal in need thereof a therapeutically effective amount of an agent that inhibits interferon-mediated gene activation of at least 3 genes selected from DDX58, G1P2, IFI35, MX1, OAS3_{—}1 and RSAD2

15. The method of claim 14, wherein said interferon is interferon alpha (IFNα).

16-17. (canceled)

18. The method of claim 14, wherein said agent inhibits at least one of HDAC, ubiquitin/proteasome, nuclear factor kB (NF-kB). Janus kinase (JAK) and I_{K}B kinase (IKK-2).

19. The method of claim 14, wherein the autoimmune disorder is type I diabetes, rheumatoid arthritis, psoriasis, psoriatic arthritis, ankylosing spondylitis, scleroderma, Reiter's Syndrome, systemic lupus erythematosus, dermatomyositis, Sjogren's syndrome, lupus erythematosus, multiple sclerosis, myasthenia gravis, asthma, encephilitis, inflammatory bowel disease, chronic obstructive pulmonary disease (COPD), arthritis, idiopathic inflammatory myopathies (IIM), dermatomyositis (DM, polymyositis (PM), includion body myositis or an allergic disorder.

20. The method of claim 19, wherein said auto-immune disease is one or more of insulin-dependent (Type I) diabetes, rheumatoid arthritis, psoriasis, myasthenia gravis, multiple sclerosis, or systemic lupus erythematosus (SLE).

21. The method of claim 20, wherein said auto-immune disease is systemic lupus erythematosus (SLE).

22. The method of claim 14, wherein said agent is a compound of Table 1 or Table 2.

23-24. (canceled)

25. A method of preventing or reducing the risk of developing an auto-immune disease or inflammatory condition in a mammal, comprising administering to a mammal in need thereof a therapeutically effective amount of an agent that inhibits interferon-mediated gene activation of at least 3 genes selected from DDX58, G1P2, IFI35, MX1, OAS3_{—}1 and RSAD2

26. The method of claim 25, wherein said interferon is interferon alpha (IFNα).

27. (canceled)

28. The method of claim 25, wherein said agent inhibits one or more of HDAC, ubiquitin/proteasome, nuclear factor kB (NF-kB). Janus kinase (JAK) and I_{K}B kinase (IKK-2).

29. The method of claim 25, wherein the autoimmune disorder or inflammatory disorder is type I diabetes, rheumatoid arthritis, psoriasis, psoriatic arthritis, ankylosing spondylitis, scleroderma, Reiter's Syndrome, systemic lupus erythematosus, dermatomyositis, Sjogren's syndrome, lupus erythematosus, multiple sclerosis, myasthenia gravis, asthma, encephilitis, inflammatory bowel disease, chronic obstructive pulmonary disease (COPD), arthritis, idiopathic inflammatory myopathies (IIM), dermatomyositis (DM, polymyositis (PM), includion body myositis or an allergic disorder.

30. The method of claim 29, wherein said auto-immune disease is one or more of insulin-dependent (Type I) diabetes, rheumatoid arthritis, psoriasis, myasthenia gravis, multiple sclerosis, or systemic lupus erythematosus (SLE).

31. The method of claim 30, wherein said auto-immune disease is systemic lupus erythematosus (SLE).

32. The method of claim 25, wherein said agent is one or more of the compounds of Table 1 or Table 2.

33-34. (canceled)


SEQUENCE LISTING (14 sequences)

SEQ ID NO: 1 - DNA, 19 bp, Artificial
  Forward PCR Primer
ggagtggctcagcgtctgt

SEQ ID NO: 2 - DNA, 19 bp, Artificial
  Reverse PCR Primer
actggctgcgacctgatct

SEQ ID NO: 3 - DNA, 20 bp, Artificial
  Forward PCR Primer
tgaaggctgctgtgtgaagt

SEQ ID NO: 4 - DNA, 25 bp, Artificial
  Reverse PCR Primer
cacacacacatgtacacaatctctc

SEQ ID NO: 5 - DNA, 22 bp, Artificial
  Forward PCR Primer
cgaactcatctttgccagtaca

SEQ ID NO: 6 - DNA, 18 bp, Artificial
  Reverse PCR Primer
gagctgctcagggacacc

SEQ ID NO: 7 - DNA, 23 bp, Artificial
  Forward PCR Primer
tggatagcaaatcctgagacaat

SEQ ID NO: 8 - DNA, 25 bp, Artificial
  Reverse PCR Primer
cctgtgtgtattccttctttttagc

SEQ ID NO: 9 - DNA, 22 bp, Artificial
  Forward PCR Primer
gggtgggttcagagtacctttt

SEQ ID NO: 10 - DNA, 26 bp, Artificial
  Reverse PCR primer
gcttcttagtatagctttgagccttc

SEQ ID NO: 11 - DNA, 25 bp, Artificial
  Forward PCR Primer
tgaactgtaagggttagtggagagt

SEQ ID NO: 12 - DNA, 24 bp, Artificial
  Reverse PCR Primer
aaataatccatttgtattgggtct

SEQ ID NO: 13 - DNA, 20 bp, Artificial
  Forward PCR Primer
ggacatcactgctctcatgc

SEQ ID NO: 14 - DNA, 22 bp, Artificial
  Reverse PCR Primer
tttatggccttcttgaaaattg